(19) United States
(12) Patent Application Publication
(10) Pub. No.: US 2025/0345135 A1
(43) Pub. Date: Nov. 13, 2025

(54) NESTING PROXIMAL LINKS FOR TABLE MOUNTED MANIPULATOR SYSTEM, AND RELATED DEVICES, SYSTEMS AND METHODS

(71) Applicant: INTUITIVE SURGICAL OPERATIONS, INC., Sunnyvale, CA (US)

(72) Inventors: Bram Gilbert Antoon Lambrecht, Redwood City, CA (US); Steven Manuel, Sunnyvale, CA (US); Kevin Lau, Fremont, CA (US)

(73) Assignee: INTUITIVE SURGICAL OPERATIONS, INC., Sunnyvale, CA (US)

(21) Appl. No.: 18/860,969

(22) PCT Filed: Apr. 28, 2023
(86) PCT No.: PCT/US2023/020322

§ 371 Date: Oct. 28, 2024

Related U.S. Application Data
(60) Provisional application No. 63/336,778, filed on Apr. 29, 2022.

(51) Int. Cl. A61B 34/35; A61B 17/00; A61B 34/30; A61B 90/57

(52) U.S. Cl. CPC A61B 34/35; A61B 90/57; A61B 2017/00477; A61B 2034/306; A61B 2090/571

(57) ABSTRACT

A teleoperable manipulator system includes a table assembly, a rail coupled to the table assembly, and first and second manipulators coupled to the rail. The table assembly includes a platform configured to support a body. The first and second manipulators include respective proximal arms coupled to the rail and respective distal portions coupled to the proximal arms and configured to support an instrument mounted thereon. The proximal arms of the first and second manipulators are translatable relative to the rail along a longitudinal dimension of the rail and are rotatable relative to the rail about a first axis perpendicular to the longitudinal dimension of the rail. The proximal arms of the first and second manipulators are positionable in a nested configuration relative to one another.

### CROSS-REFERENCE TO RELATED APPLICATION

This application claims priority to U.S. Provisional Application No. 63/336,778 (filed Apr. 29, 2022), titled “NESTING PROXIMAL LINKS FOR TABLE MOUNTED MANIPULATOR SYSTEM, AND RELATED DEVICES, SYSTEMS AND METHODS” the entire contents of which are incorporated by reference herein.

### FIELD

Aspects of this disclosure relate generally to table mounted manipulator systems. In particular, aspects of the disclosure relate to link configurations of manipulators mounted to a table, such as a medical system table for supporting a patient. Related devices, systems, and methods also are disclosed.

### INTRODUCTION

Computer-assisted manipulator systems (“manipulator systems”), sometimes referred to as robotically assisted systems or robotic systems, can include one or more manipulators that can be operated with the assistance of an electronic controller (e.g., computer) to move and control functions of one or more instruments when coupled to the manipulators. A manipulator generally includes mechanical links connected by joints. An instrument is removably couplable to (or permanently coupled to) one of the links, typically a distal link of the plural links. The joints are operable to cause the links to move (i.e., rotate and/or translate) relative to one another, imparting various degrees of freedom to the manipulator to enable the manipulator to move the instrument relative to a worksite. The manipulators of a manipulator system can be used to transmit a variety of forces and torques to the instruments to perform various procedures, such as medical procedures or non-medical procedures (e.g., industrial procedures). The link to which the instrument is couplable or coupled (e.g., an instrument carriage) includes drive outputs to interface with and mechanically transfer driving forces to corresponding drive inputs of the instrument to control degrees of freedom of motion and/or other functions of the instrument.

In some computer-assisted manipulator systems, the manipulators are attached to a manipulator support structure (e.g., a patient side cart) that is separate from a support structure that supports a patient or workpiece. In other manipulator systems, the manipulators are attached directly to the support structure that supports the patient or workpiece, e.g., to an operating table. This support structure that supports the patient or workpiece can be referred to herein as a “table assembly” or “table” to simplify the description. Manipulator systems in which the manipulators are mounted to the table assembly can be referred to herein as table-mounted manipulator systems.

Table-mounted manipulator systems pose certain challenges. The space around a table assembly may need to be occupied with various pieces of equipment and/or personnel during the performance of various tasks that make up a procedure (e.g., a medical procedure). Moreover, the space constraints around the table can vary depending on the procedure being performed, with some tasks (such as transferring a patient to the table, draping manipulators, etc.) benefiting from or being facilitated by a large amount of open space around the table. In manipulator systems including movable patient-side carts, such open space around the table can be obtained by moving the patient-side cart away from the table intended to support the patient. However, in table-mounted manipulator systems, moving the manipulators out of the way when space is desired around the table poses challenges as the manipulators are either affixed to the table or at least not practical to remove from the table during a particular stage of a medical procedure. Thus, it can be challenging to avoid interference between the manipulators and the other entities in the space around the table in a table-mounted manipulator system.

By way of example, while transferring a patient from a patient-carrying gurney to the table, it can be desirable for a long side of the table to be substantially free of obstructions so as to allow the gurney to be placed flush with the table to ease the transfer of patient from gurney to table. But when manipulators are mounted to this long side of the table, the needed open space can be difficult to achieve as the manipulators might get in the way. As another example, while preparing a patient on the table, a number of personnel and equipment may need to be close to the patient and can occupy much of the space around the table and therefore the manipulators mounted to the table can get in the way of personnel and hinder the preparations. As another example, covering the manipulators with a sterile drape can be easier when there is a substantial amount of free space around the manipulator, but when the manipulators are attached to the table assembly it can be difficult to obtain such space due to the proximity of the table.

Accordingly, a need exists for improved table mounted manipulator systems, in particular systems with improved configurations of manipulators and how they are mounted to the table and relative to one another.

### SUMMARY

Various embodiments of the present disclosure may solve one or more of the above-mentioned problems and/or may demonstrate one or more of the above-mentioned desirable features. Other features and/or advantages may become apparent from the description that follows.

Some embodiments of the present disclosure relate to a teleoperable manipulator system. The system can include a table assembly, a rail coupled to the table assembly, and first and second manipulators coupled to the rail. The table assembly can include a platform configured to support a body. The first and second manipulators can include respective proximal arms coupled to the rail and respective distal portions coupled to the proximal arms and configured to support an instrument mounted thereon. The proximal arms of the first and second manipulators can be translatable relative to the rail along a longitudinal dimension of the rail and rotatable relative to the rail about a first axis perpendicular to the longitudinal dimension of the rail. The proximal arms of the first and second manipulators can be positionable in a nested configuration relative to one another.

In some embodiments, the system can include a table assembly, a rail coupled to the table assembly, and a plurality of manipulators coupled to the rail. The table assembly can include a platform configured to support a body. Each manipulator can include a proximal arm coupled to the rail and a distal portion coupled to the proximal arm and configured to support an instrument mounted thereon. The proximal arm of each manipulator can be translatable relative to the rail along the longitudinal dimension of the rail and rotatable relative to the rail about a first axis perpendicular to the longitudinal dimension. The proximal arms of at least two of the plurality of manipulators can have staggered heights relative to the rail along a direction perpendicular to lateral and longitudinal dimensions of the rail.

In some embodiments, the system can include a table assembly, a rail coupled to the table assembly, and a plurality of manipulators coupled to the rail. The table assembly can include a platform configured to support a body. Each manipulator can include a proximal arm coupled to the rail and a distal portion coupled to the proximal arm and configured to support an instrument mounted thereon. The proximal arm of each manipulator can be translatable relative to the rail along a longitudinal dimension of the rail and rotatable relative to the rail about a first axis perpendicular to a longitudinal dimension of the rail. In a state of the proximal arms of the manipulators being positioned adjacent one another at a same end portion of the platform, the proximal arms of the manipulators can be oriented at respective outward angles relative to the longitudinal dimension of the rail that are all equal to or greater than 180 degrees.

In some embodiments, the system can include a table assembly, a rail coupled to the table assembly, and first and second manipulators coupled to the rail. The table assembly can include a platform configured to support a body. Each manipulator can include a proximal arm coupled to the rail and a distal portion coupled to the proximal arm and configured to support an instrument mounted thereon. The first and second manipulators can be positionable between a stowed position and a plurality of deployed positions. In the stowed position, the first and second manipulators can each be positioned under a first end portion of the platform and do not protrude beyond lateral or longitudinal dimensions the platform.

In some embodiments, the system can include a table assembly, a rail coupled to the table assembly, and first and second manipulators movably coupled to the rail. The table assembly can include a platform configured to support a body, the platform elongate along a longitudinal dimension. The rail can extend parallel to the longitudinal dimension. The first and second manipulators can be moveable in translation along the rail and each includes a proximal arm coupled to the rail and a distal portion coupled to the proximal arm and configured to hold a medical tool. The first and second manipulators can be arrangeable such that at least the proximal arms thereof are in a nested configuration.

The proximal arms of the first and second manipulators can be positionable in a nested configuration relative to one another in a deployed state. In some embodiments, the proximal arms can be oriented at respective outward angles of at least 180 degrees relative to the rail and overlap one another in a given direction in the nested configuration. The given direction can be parallel to the lateral dimension of the rail. In some embodiments, the proximal arm of the first manipulator is offset from the axis perpendicular to the longitudinal dimension of the rail. In some embodiments, a coupling portion of the first a manipulator that rotatably couples the proximal arm to the rail can include a bend, and the bend can be between the axis and the proximal arm.

In some embodiments, the first and second manipulators further can include respective coupling portions that rotatably couple the proximal arms to the rail, and the coupling portion of the first manipulator can have a longer height dimension than the coupling portion of the second manipulator. The first and second manipulators can further include respective coupling portions that rotatably couple the proximal arms to the rail, and the coupling portion of the first manipulator can allow the proximal arm of the first manipulator to rotate around the first axis and around a second axis parallel to the longitudinal dimension of the rail.

In the nested configuration and in a deployed state, the distal portions of the first and second manipulators can be positioned higher than the respective proximal arms of the first and second manipulators. The distal portions of the first and second manipulators can include instrument holders, and in the nested configuration the instrument holders of the first and second manipulators can be positioned higher than the platform. In the nested configuration the proximal arms can be positioned adjacent one another at one of the end portions of the platform.

The platform can include an assembly of a plurality of sections movable relative to one another to change a configuration of the platform between a plurality of configurations. The plurality of sections can include a first end section, one or more middle sections, and a second end section consecutively positioned along a longitudinal dimension of the platform, and each of the first and second end portions can be independently pivotable relative to the one or more middle sections.

In some embodiments, the system can further include one or more first carriages, each first carriage movably coupling one of the proximal arms of the first and second manipulators to the rail such that the proximal arm is translatable relative to the rail along a longitudinal dimension of the rail. In some embodiments, the system can further include a control system operably coupled to drive the first and second manipulators to position the proximal arms in the nested configuration.

The table assembly can include a support column supporting the platform assembly, and the platform can be tiltable relative to the support column. The rail can be configured to tilt along with the platform relative to the support column.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1A is a schematic side view of an embodiment of a table-mounted manipulator system in a first state.

FIG. 1B is a schematic side view of the table-mounted manipulator system of FIG. 1A in a second state.

FIG. 2 is a schematic end view of the table-mounted manipulator system of FIG. 1A.

FIG. 3A is a perspective view of the table-mounted manipulator system of FIG. 1A, with only a portion of the manipulators shown.

FIG. 3B is a perspective view of the table-mounted manipulator system of FIG. 1B, with only the portion of the manipulators shown.

FIG. 4A is a perspective view of an embodiment of a table-mounted manipulator system in a first state, with only a portion of the manipulators shown.

FIG. 4B is a perspective view of the table-mounted manipulator system of FIG. 4A in a second state.

FIG. 4C is a perspective view of the table-mounted manipulator system of FIG. 4A in a third state.

FIG. 4D is a perspective view of the table-mounted manipulator system of FIG. 4A in a fourth state.

FIG. 5 is a perspective view of an embodiment of a table-mounted manipulator system in a first state.

FIG. 6 is a perspective view of the table-mounted manipulator system of FIG. 5 in a second state.

FIG. 7A is a side view of the table-mounted manipulator system of FIG. 5 in a third state.

FIG. 7B is a side view of the table-mounted manipulator system of FIG. 5 in a fourth state and illustrating a fifth state in dashed lines.

FIG. 7C is a side view of the table-mounted manipulator system of FIG. 5 in a sixth state and illustrating a seventh state in dashed lines.

FIG. 7D is a side view of the table-mounted manipulator system of FIG. 5 in an eight state and illustrating a ninth state in dashed lines.

FIG. 8A is a perspective view of an embodiment of a table-mounted manipulator system in a first state, with only a portion of the manipulators shown.

FIG. 8B is a perspective view of the table-mounted manipulator system of FIG. 8A in a second state.

FIG. 9 is a top view of the table-mounted manipulator system of FIG. 5 with manipulators in a deployed and nested configuration, with a gurney adjacent the table-mounted manipulator system.

FIG. 10 is a perspective view of the table-mounted manipulator system of FIG. 5 with manipulators in the deployed and nested configuration of FIG. 9.

FIG. 11 is a perspective view of a table-mounted manipulator system.

### DETAILED DESCRIPTION

As discussed above, there can be certain challenges arising from having manipulators attached to a table assembly in a table-mounted manipulator system, such as challenges associated with the manipulators, personnel, and/or a table obstructing or interfering with one another during various stages of a medical procedure.

In various embodiments described herein, table-mounted manipulators can be movable between a stowed configuration in which the manipulators are compacted or folded (e.g., while not in use), and a variety of deployed configurations in which the manipulators are at least partially unfolded (e.g., for use in a procedure). One way to avoid the manipulators becoming an obstruction or otherwise interfering with a task is to place the manipulators in a stowed state or configuration during a stage that requires more space around the table. In the stowed state, the manipulators are generally compacted (e.g., folded) and placed in a stowed location, such as under a platform of the table assembly. However, stowing of the manipulators to make sufficient space for a task is not always feasible, as some tasks may require or benefit from the manipulators being in a deployed configuration during the task. Moreover, even when the task being performed does not require the manipulators to be in a deployed configuration, in some circumstances having the manipulators positioned in a stowed configuration during the task can be undesirable. For example, when manipulators are prepared for a procedure, they are placed in a sterile condition, such as by covering exposed manipulator portions with a sterile drape. Conversely, stowing the manipulators can compromise the sterile condition of the manipulators, which can have various undesired consequences. The stowing of the manipulators can compromise the sterility of the manipulators because the stowed location (e.g., under the table) is generally not within the sterile field established around the table. The sterile field is a region in which any exposed surfaces of objects in the region are maintained in a sterile condition (e.g., a condition substantially free from contaminants, such as biological pathogens, dusts, oils, etc.) and non-sterile surfaces are covered by a sterile barrier. Because manipulators positioned in a stowed configuration can compromise their sterility, the manipulators would need to be placed in a sterile configuration (e.g., covered with a sterile drape) after completion of the task for which space around the table was needed and once the manipulators have been moved from the stowed state into the sterile field. But placing the manipulators in a sterile condition (e.g., draping) of the manipulators after completion of the task is undesirable in some circumstances. For example, when the task involves the transferring of a patient to the table and/or preparation of the patient on the table, draping the manipulators after completion of the task will cause a delay between when the patient is ready and when subsequent tasks of the overall medical procedure can be performed. Such a delay is generally undesirable, as once the patient has been prepared for the procedure (e.g., transferred to the table) it is generally desirable to proceed with and finish the overall medical procedure as quickly as is feasible. On the other hand, prior to bringing patient to the procedure room and prepping the patient for the procedure, time is less constrained and delays are generally more acceptable. Thus, it is generally desired to perform as many tasks as possible prior to the preparation of the patient so as to minimize the time the patient is on the table. Another reason it may be desirable to drape the manipulators prior to transferring the patient to the table it that it is generally easier to drape the manipulators when the patient is not present, as the personnel may have more room to maneuver at that time. Another reason to drape the manipulators prior to transferring the patient to the table is to avoid having non-sterile objects in the vicinity of a sterile patient, even if only temporarily (e.g., while they are in the process of being draped). Thus, even when stowing of the manipulators to free up space is possible, the stowing of the manipulators may not always be an acceptable option to free up space for a given stage of an overall procedure.

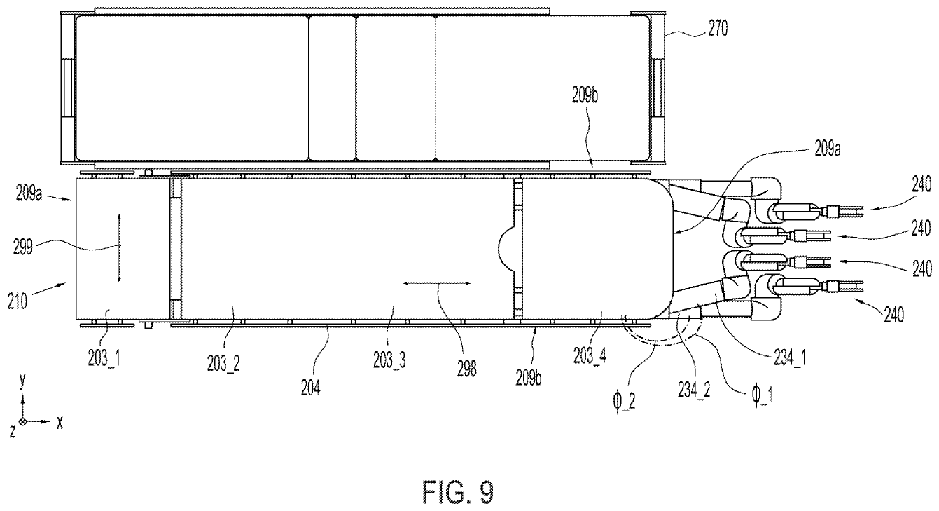

Another way to mitigate some of the challenges noted above is to configure the system to allow relative movement between the manipulators and the table, such as by movably attaching the manipulators to a rail coupled to the table assembly. This can allow the manipulators to be moved along the rail towards one end of the table, thus clearing up some space in a middle portion of the table. However, even with the ability to move the manipulators, the manipulators can nevertheless obstruct certain tasks. For example, as noted above, transferring a patient to the table may benefit from having an entire longitudinal side of the table be free of obstruction so that a gurney can be positioned flush with the longitudinal side of the table. When multiple manipulators coupled to the same rail are moved to one end of the table, at least one of the manipulators is generally going to remain at least partially along a longitudinal side of the table and thus potentially interfere with equipment (e.g., the gurney) and/or personnel. This occurs because the proximal link of one manipulator (i.e., the link that is coupled to the rail) can block another manipulator and prevent the other manipulator from being moved fully out of the way. For example, consider the system illustrated in FIG. 11, which includes a platform assembly 10 supported by a support column 2, a rail 20 coupled to the platform assembly 10, and two manipulators 40_1 and 40_2 movably coupled to the rail 20 (only a portion of the most proximal link of each manipulator is depicted). In the system of FIG. 9, the manipulators 40_1 and 40_2 are translatable along a direction of a longitudinal dimension 8 of the platform assembly 10, and the proximal links of the manipulators 40_1 and 40_2 are also rotatable coupled to the rail to allow rotation of the links around vertical rotation axes 7.1 and 7.2. The manipulators 40_1 and 40_2 can be in a deployed configuration wherein the manipulators are at least partially unfolded. In the deployed configuration, portions of the manipulators 40_1 and 40_2 can extend generally upward and/or away from the rail 20. As shown in FIG. 11, when the manipulator 40_1 is positioned at an end of the rail 20, the proximal link of the manipulator 40_1 can be rotated to an angle φ_1 that is more than 180 degrees relative to the rail 20 such that distal portions of the manipulator 40_1 can be moved away from the longitudinally extending side (i.e., a side extending along x-directions in FIG. 11, or along dimension 8 of the platform assembly) of the platform assembly 10 around the end of the platform assembly 10 to be positioned along the laterally extending side (i.e., a side extending along y-directions in FIG. 11, or along dimension 9 of the platform assembly) of the platform assembly, while remaining in a deployed configuration. In other words, the manipulator 40_1 can be positioned in a deployed configuration without blocking portions of the longitudinal side of the platform assembly 10. However, as shown in FIG. 11, the proximal link of the manipulator 40_2 cannot be rotated relative to the rail 20 as far as the manipulator 40_1 could be because the proximal link of the manipulator 40_1 blocks the proximal link of the manipulator 40_2. As a result, the manipulator 40_2 is only rotatable to an angle φ_2 that is less than 180 degrees while deployed and therefore distal portions of the manipulator 40_2 cannot be moved fully around the end of the platform assembly 10 to be positioned along the lateral side of the platform assembly 10. (The angles φ_1 and φ_2 are outward facing angle as illustrated in FIG. 11.) Thus the manipulator 40_2 continues to protrude laterally into the space alongside the longitudinally extending side of the platform assembly 110 and can interfere with certain tasks, such as patient transfer. Note that it is generally not feasible to avoid this issue of one of the manipulators continuing to protrude beyond the longitudinal side of the platform by moving the manipulators to opposite ends of the platform assembly from one another instead of moving them towards the same end as one another. One reason why it is generally not feasible to move the manipulators to opposite ends of the table as one another is that one end of the table (often the head end) is generally reserved for various equipment and personnel, and therefore there may not be sufficient room for a manipulator to be moved to the head end of the table. Moreover, various lines (e.g., IV lines, irrigation/suction tubes, electrical cords and/or cables, etc.) may need to be routed to the patient and/or equipment around the table. These lines are generally routed around an end of the table that is opposite from where the manipulators are expected to be predominantly located so as to avoid or minimize collision between the manipulators and the lines when the manipulators are returned to their working positions. However, routing the lines along an end of the table that is away from the manipulators is not possible if there are manipulators positioned at the head end of the table and manipulators positioned at the foot end of the table. Thus, when the manipulators are positioned at opposite ends of the table from each other during a task such as patient transfer, it can be difficult to route the lines in a manner that does not result in collision with one of the manipulators when the manipulators are returned to their operational positions (e.g., near a middle of the table).

Thus, to address the above-described challenges with table-mounted manipulator systems, various embodiments disclosed herein contemplate a table-mounted manipulator system including a table assembly and a rail that is coupled to the table assembly. The table assembly includes a platform to support a patient or workpiece. The rail supports two or more manipulators, which are translatable along the rail. The manipulators are configured such that they can be placed in a nested configuration. The nested configuration can allow the manipulators coupled to the rail to be positioned fully or partially out of the way of the longitudinally extending side of the table assembly, thus freeing up space for various tasks. In particular, in some embodiments, the nested configuration includes a configuration in which proximal links of the manipulators, which are coupled to the rail, are oriented at angles of 180 degrees or greater relative to the rail (i.e., relative to a longitudinal dimension thereof) and overlap one another in a given direction (the given direction can vary from one embodiment to another). (The angles referred to herein are outward facing relative to the rail, as illustrated in the Figures and described in greater detail below.) In some embodiments, in the nested configuration the proximal links of the manipulators overlap one another in a vertical direction. As used herein, “horizontal” refers to a direction parallel to a horizontal plane, which is a plane defined by (i.e., parallel to) lateral and longitudinal dimensions of the rail assembly, and “vertical” refers to a direction perpendicular to the horizontal plane. In some embodiments, the vertical nesting of the proximal links is achieved by staggering the relative heights of the respective proximal links of the manipulators relative to the rail. This vertically overlapping nested configuration can allow, in a state in which the manipulators coupled to the rail to are positioned at one end portion of the platform assembly (e.g., a foot end), distal portions of the manipulars to be swung around the end of the platform such that the manipulators are moved fully away from the longitudinally extending side of the table assembly to positions along the laterally extending side (end sides) of the table assembly while in a deployed configuration. Thus, in some embodiments disclosed herein the longitudinally extending sides of the table assembly can be cleared of obstructions while the manipulators are deployed, thus facilitating various tasks that benefit from or require such free space, such as transferring a patient from a gurney to the table. In other embodiments, the proximal links of the manipulators can overlap in a horizontal direction in the nested configuration, which can leave some portions of the manipulators protruding past the longitudinally extending side of the platform assembly, but which can nevertheless provide sufficient free space for certain tasks in some circumstances. The nested configuration can be used in both deployed and stowed states. In some embodiments, in a deployed state of the manipulators and in the nested configuration, the manipulators are configured to remain within the sterile field, and thus the manipulators can be draped prior to the performance of a task for which extra space is desired (e.g., patient transfer) without having their sterility compromised as a result of being moved out of the way for the task.

In some embodiments, each of the manipulators further includes a coupling portion coupling proximal links of the manipulators to the rail, with the coupling portion including one or more joints to provide for rotation of the proximal link relative to the rail. The coupling portions and proximal link of a first manipulator forms an L-shape portion configured to allow the proximal link of another manipulator to nest adjacent to the proximal link of the first manipulator in a space defined between the two legs of the L-shape, with the proximal links of the two manipulators overlapping in a given direction. In some embodiments in which the proximal links overlap one another in a vertical direction in the nested configuration, in a neutral state of the first manipulator the coupling portion of the first manipulator extends vertically from the rail and the proximal link extends horizontally from the coupling portion, whereas the coupling portion of a second manipulator is at a same height as the proximal link coupled thereto. Thus, due to the vertical extent of the coupling portion of the first manipulator, the proximal link of the first manipulator is positioned at a lower height than the proximal link of the second manipulator. This difference in height allows the proximal links to vertically overlap one another without collision. In other embodiments, in which the proximal links are nested horizontally, the coupling portion of the first manipulator extends horizontally from the rail in a first direction and the proximal link of the first manipulator extends horizontally from the coupling portion in a second direction, perpendicular to the first direction, forming a horizontally oriented L-shaped portion. This allows the proximal link of a second manipulator to be positioned adjacent to and horizontally overlapping with the proximal link of the first manipulator.

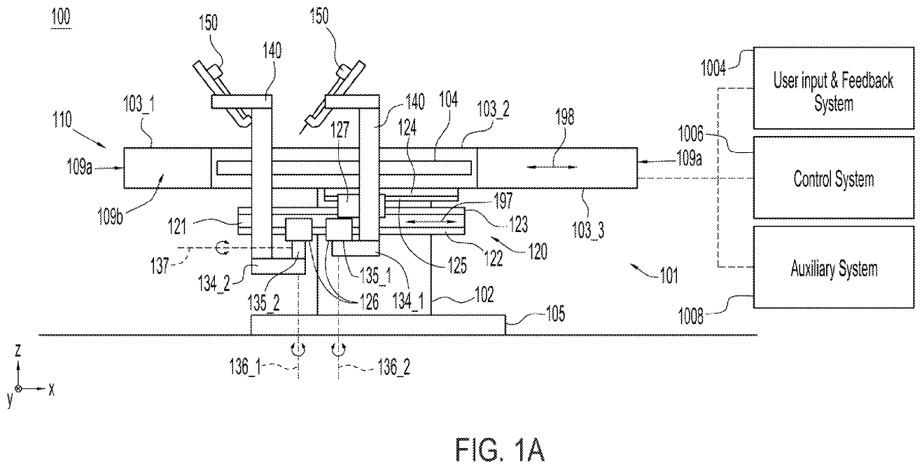

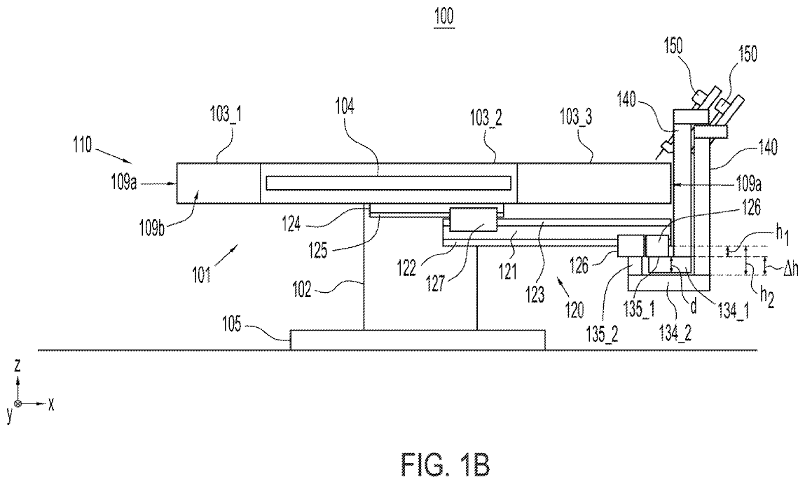

Turning now to FIGS. 1-8B, embodiments of table-mounted manipulator systems will be described.

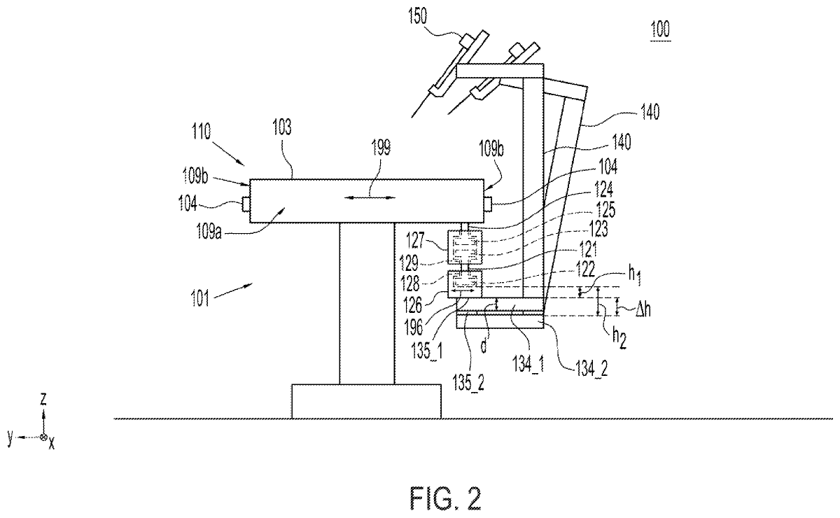

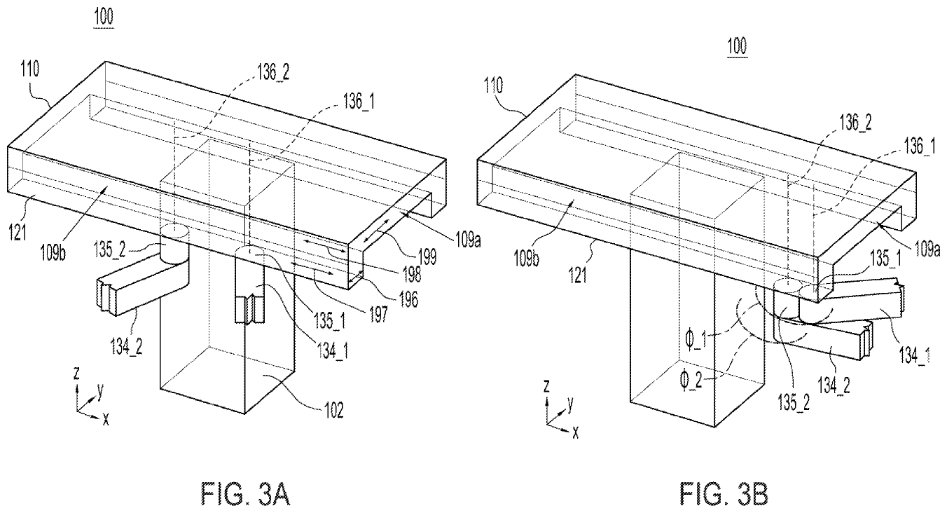

FIGS. 1A-3B schematically illustrate an embodiment of a table-mounted manipulator system 100 (“system 100”). FIGS. 1A and 1B illustrate side views of the system 100 in two different states, FIG. 2 illustrates an end view of the system 100, and FIGS. 3A and 3B illustrate portions of the system 100 in schematic perspective with other portions omitted for clarity. As shown in FIGS. 1A-2, the system 100 includes a table assembly 101, at least one rail assembly 120 coupled to the table assembly 101, and two or more manipulators 140 coupled to the rail assembly 120. Each manipulator 140 can be configured to carry one or more instruments 150, which can be removably or permanently mounted thereon. As shown in FIG. 1A, the system 100 also can include a control system 1006, a user input and feedback system 1004, and/or an auxiliary system 1008. In some embodiments, the system 100 is configured as a computer-assisted, teleoperable medical system, in which case table assembly 101 can be configured to support a patient (not shown) and the instruments 150 can be medical instruments. The system 100 in this configuration may be usable, for example, to perform any of a variety of medical procedures, such as surgical procedures, diagnostic procedures, imaging procedures, therapeutic procedures, etc. Moreover, the system 100 when configured as a teleoperable medical system need not necessarily be used on a living human patient. For example, a non-human animal, a cadaver, tissue-like materials used for training purposes, and so on, can be supported on the table assembly 101 and worked on by system 100. In other embodiments, the system 100 is configured as a computer-assisted teleoperable system for use in non-medical contexts, in which case the table assembly 101 can be configured to support an inanimate workpiece (something being manufactured, repaired, tested, etc.) and the instruments 150 can be non-medical instruments, such as industrial instruments.

As shown in FIG. 1A, the table assembly 101 includes a platform assembly 110 (also “platform 110”) configured to support the patient or inanimate workpiece, a support column 102 coupled to and supporting the platform assembly 110, and a base 105 coupled to the support column 102. The base can be configured to contact the ground or other surface upon which the table assembly 101 rests to provide stability for the table assembly 101. In some embodiments, the base 105 is omitted. In some embodiments, the base 105 includes mobility features, such as wheels, tracks, or other such features (not shown), to allow movement of the table assembly 101 along the ground or other surface. In FIGS. 1A-2, the support column 102 is illustrated as a single vertical columnar part to simplify the discussion, but the support column 102 could take any desired shape and could include any number of parts. For example, the support column 102 can include horizontal support structures (not illustrated) such as beams, rails, etc. to couple the platform assembly 110 to a vertical portion of the support column 102. Moreover, in various embodiments, the support column 102 can be telescoping and configured to extend and contract in height.

The platform assembly 110 includes one or more platform sections 103 to support the patient or workpiece. The platform sections 103 each have a support surface configured to contact and support the patient or workpiece. In some embodiments multiple platform sections 103 are used and the platform sections 103 are arranged in series to support different portions of the patient or workpiece. For example, in the embodiment illustrated in FIG. 1A, the platform assembly 110 includes a first end section 103_1, one or more middle sections 103_2, and a second end section 103_3, with the one or more middle sections 103_2 being arranged between the two end sections 103_1 and 103_3. In some embodiments, the first end section 103_1 can be configured to support a head of the patient, the second end section 103_3 can be configured to support the feet and/or legs of the patient, and the one or more middle sections 103_2 can be configured to support a torso and/or other portions of the patient. For convenience, the side of the platform assembly 110 that is near the first end section 103_1 (e.g., a left side in the orientation shown in FIG. 1A) will be referred to herein as a “head” of the platform assembly 110 (or “head side” or “head end”) and the side of the platform assembly 110 that is near the second end section 103_3 (e.g., a right side in the orientation shown in FIG. 1A) will be referred to herein as a “foot” of the platform assembly 110 (or “foot side” or “foot end”), but this is merely an arbitrary convention chosen herein for convenience of description and is not intended to limit the configuration or usage of the table assembly 101 (e.g., a head of a patient could be positioned at the “foot” side of the platform assembly 110 if desired, and vice versa). The relative positions of two components or of two portions of a single component may also be described using “head” and “foot” (e.g., a “head end” and a “foot end” of a rail 121) with “head” referring to the component or portion that is relatively closer to the head end of the table assembly 110 and foot referring to the component or portion that is relative closer to the foot end of the table assembly 110. In other embodiments, different numbers and arrangements of platform sections 103 are used, including one, two, four, or more platform sections 103. In some embodiments, one or more of the platform sections 103 can be movable relative to other platform sections 103 and/or relative to the support column 102. For example, in some embodiments, some or all of the platform sections 103 are coupled to adjacent platform sections 103 and/or to the support column 102 by rotatable joints such that at least some of the platform sections 103 can tilt relative to one another and/or relative to the support column 102. The platform assembly 110 can also be movable as a whole relative to the support column 102, as described in greater detail below.

The platform assembly 110 has a longitudinal dimension 198 (see FIG. 1), a lateral dimension 199 orthogonal to the longitudinal dimension 198 (see FIG. 2), and a thickness or height dimension (not labeled) orthogonal to both the longitudinal and lateral dimensions. As used herein, the longitudinal dimension 198 refers to a dimension of greatest extent of the platform assembly 110 when all of the platform sections 103 of the platform assembly are fully extended and all are oriented with their support surfaces roughly aligned in a same plane with one another (or when as close to this state as possible) so as to collectively form a combined support surface that is substantially planar with potentially small gaps between adjacent platform sections 103. The longitudinal direction extends in a heat-to-foot and vice-versa direction of the platform assembly 110. In general, the longitudinal and lateral dimensions 198 and 199 of the platform assembly 110 and the support surfaces of the platform sections 103 are oriented roughly parallel to the ground or other surface on which the table assembly 101 is supported when the platform assembly 110 is in a neutral configuration. For example, in FIGS. 1A-2 the longitudinal dimension 198 is parallel to the x-direction and the lateral dimension 199 is parallel to the y-direction, with the x- and y-directions being parallel to the ground or other surface the table assembly 101 rests upon. Thus, in FIGS. 1A-2, the thickness dimension is parallel to the z-direction, which is perpendicular to the ground or other surface. However, one of ordinary skill in the art would understand that the platform assembly 110 as a whole and/or individual platform sections 103 thereof do not necessarily have to be parallel to the ground, and that one or both of the longitudinal and/or lateral dimensions 198 and 199 can be tilted relative to the ground in various configurations through which the platform assembly 110 and/or platform section 103 can be movable, including in a neutral configuration in some cases.

At least one of the platform sections 103 is directly coupled to and supported by the support column 102. The remaining platform sections 103 can be coupled directly to the support column 102 or they can be coupled indirectly to the support column 102 via a chain of one or more intervening platform sections 103. For example, in some embodiments a main platform section 103 (e.g., a middle section 103_2) is coupled to and directly supported by the support column 102 and the others of the platform sections 103 (e.g., end sections 103_1 and 103_3) are coupled to the main platform section 103 or to another platform section 103. As another example, in some embodiments multiple platform sections 103 (all in some embodiments) are coupled directly to the support column 102 and not to another platform section 103.

In some embodiments, some (all, in some cases) of the above-described parts of the table assembly 101 can be movable relative to one another. For example, in some embodiments the platform assembly 110 as a whole can be moved relative to the support column 102, such as by tilting around a horizontal axis, swiveling around a vertical axis, translating vertically along the support column 102, translating horizontally relative to the support column 102, and so on. In some embodiments, such movement of the platform assembly 110 as a whole can be provided by one or more joints that couple a main platform section 103 (e.g., a middle section 103_2) to the support column 102. Furthermore, as already noted above, individual platform sections 103 can be movable relative to one another and relative to the support column 102 as well, which can be facilitated by joints coupling the platform sections 103 to the support column 102 or to adjacent platform sections 103.

In some embodiments, the platform assembly 110 also includes one or more accessory rails 104. The accessory rails 104 can be configured to receive accessory devices removably mounted thereon, such as arm supports, leg supports, body restraints, width extensions, various clamps for surgical retractors and device holders. In some embodiments, the accessory rails 104 adhere to industry standard specifications familiar to those of ordinary skill in the art to allow compatibility with accessory devices compliant with the standard. The accessory rails 104 can be attached to longitudinally extending sides of one or more of the platform sections 103. One or more openings can be defined between an accessory rail 104 and the side of the platform section 103 to which the accessory rail 104 is attached and portions of accessories mounted to the accessory rail 104 can be inserted through the openings.

As noted above, the system 100 includes two or more manipulators 140. FIGS. 1A-2 illustrate two manipulators 140, but any number of manipulators 140 can be included (e.g., one, two, three, or more manipulators 140 coupled to each rail assembly 120). A manipulator 140 can include a kinematic structure of links coupled together by one or more joints. The manipulator 140 is movable through various degrees of freedom of motion provided by the joints, thus allowing an instrument 150 mounted thereon to be moved relative to the worksite. For example, some joints can provide for rotation of links relative to one another, other joints can provide for translation of links relative to one another, and some can provide for both rotation and translation. Some or all of the joints can be powered joints, meaning a powered drive element can control movement of the joint through the supply of motive power. Such powered drive elements can include, for example, electric motors, pneumatic or hydraulic actuators, etc. Additionally, some joints can be unpowered joints. The specific number and arrangement of links and joints is not limited. The more links and joints are included, the greater the degrees of freedom of movement of the manipulator 140.

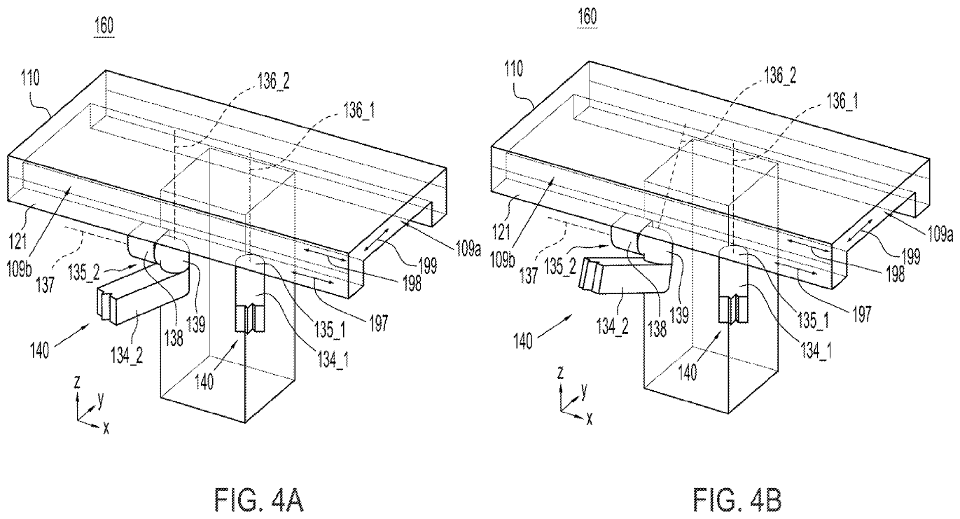

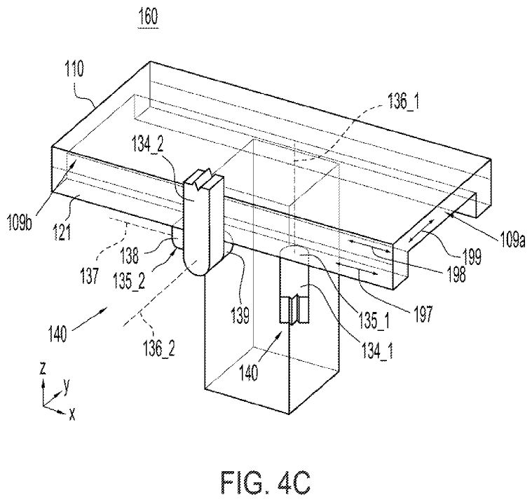

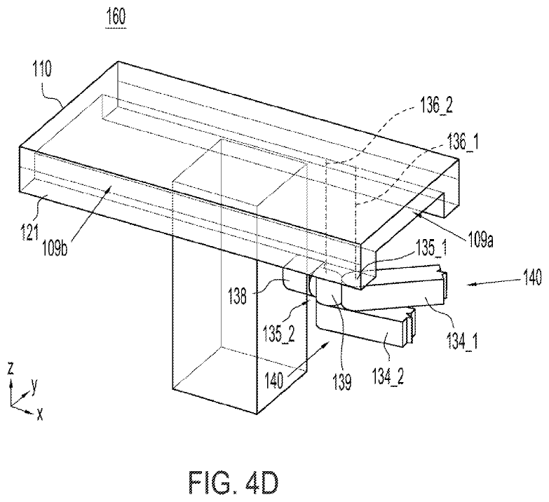

As shown in FIGS. 1A-2, a proximal end portion of each manipulator 140 is movably coupled to the table assembly 101 via a rail assembly 120, as described in further detail below. The proximal end portion of each manipulator 140 can include a proximal arm 134 (e.g., proximal arms 134_1 and 134_2) and a rail coupling portion 135 (also referred to as “coupling portion”, e.g., coupling portions 135_1 and 135_2). (FIGS. 3A and 3B illustrate the proximal arm 134 and a rail coupling portion 135 of the manipulators 140, but omit other portions of the manipulators 140 from the view for clarity.) The proximal arm 134 includes one or more proximal links. The rail coupling portion 135 is coupled to the rail assembly 120 (specifically, to a carriage 126, described in greater detail below) and the proximal arm 134 extends from the rail coupling portion 135. In some cases, the rail coupling portion 135 can be part of the proximal arm 134, such as the rail coupling portion 135_1 illustrated in FIGS. 1A-3B. In other cases, the rail coupling portion 135 can be a separate component coupled with the proximal arm 134, such as the rail coupling portion 135_2 illustrated in FIGS. 1A-3B. In a neutral state of the platform assembly 110 (e.g., the platform 110 is parallel to the ground or other supporting surface) and a neutral state of the proximal arm 134, the proximal arm 134 extends horizontally from the rail coupling portion 135, as shown in FIGS. 1A-3B. However, in some embodiments, at least one of the proximal arms 134 can be capable of being oriented in other directions in other states, such as in the embodiments described below in relation to FIGS. 4A-4B. The manipulators 140 can each include additional links (not labeled) which are coupled to and supported by the proximal arm 134.

Each coupling portion 135 includes one or more joints that enable motion of the proximal arm 134 (and hence motion of more distal portions of the manipulator 140) relative to the rail assembly 120. In particular, the rail coupling portion 135 of each manipulator 140 includes at least a first rotational joint that can provide rotation of the proximal arm 134 around a first rotation axis 136 (e.g., axes 136_1 and 136_2, see FIGS. 1A, 3A, and 3B), which is aligned with a vertical direction in the neutral state of the platform assembly 110 and a neutral state of the proximal arm 134.

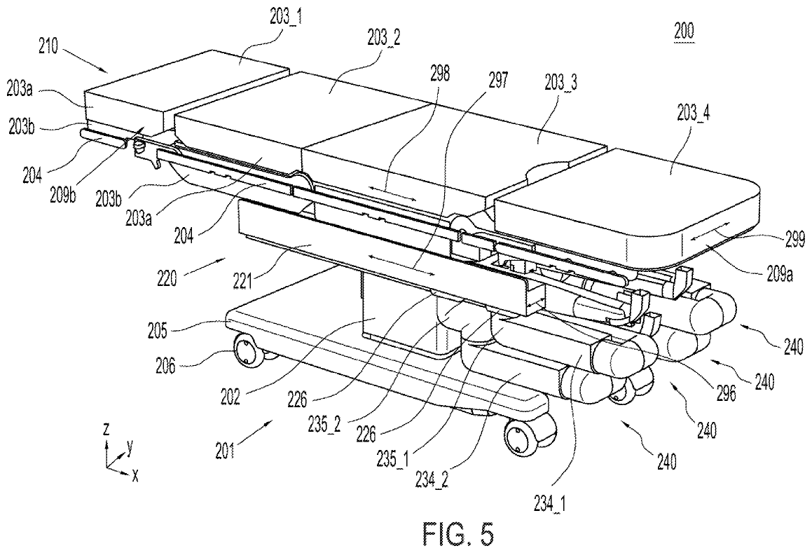

The manipulators 140 are configured such that they can be placed in a nested configuration, wherein portions of one manipulator 140 can be nested within portions of another manipulator 140. The nested configuration can be used in both deployed and stowed states of the manipulators 140. The deployed state of a manipulator 140 includes any state in which the manipulator 140 is removed from a stowed position (e.g., under the platform 110) and at least partially unfolded such that a distal end portion of the manipulator 140, which can include an instrument holder that is configured to carry an instrument 150, is positioned at or above a predetermined height, such as a height of the rail 121, a height of a bottom surface of the platform assembly 110, a height of a top surface of the platform assembly 110, etc. For example, in some embodiments, the aforementioned the predetermined height includes a boundary of the sterile field, and because the distal ends of the manipulators 140 are located above the predetermined height when deployed, the distal end portions of the deployed manipulators 140 are generally located within the sterile field. In contrast, manipulators 140 in a stowed state are generally compacted (folded) and arranged at stowed locations that are outside of the sterile field (e.g., under platform assembly 110) (see, for example, the manipulators 240 in FIG. 5 as one example of a stowed state). In some embodiments the manipulators 140 remain within the sterile field while in the deployed configuration. Thus, in some embodiments, when the manipulators 140 are deployed and in the nested configuration, the manipulators 140 remain within the sterile field. The manipulators 140 can also be placed in the nested configuration when stowed. The nested configuration of manipulators 140 allows the manipulators 140 to be positioned fully or partially out of the way of a longitudinal side 109b of the platform assembly 110, even when deployed, thus freeing up space along the longitudinal side 109b for various tasks.

In some embodiments, the nested configuration includes a configuration in which each of the proximal arms 134 (e.g., a most proximal link thereof) is oriented at an angle of 180 degrees or more relative to the rail 121, as shown in FIG. 3B. In other words, in the nested configuration the proximal arms 134 are oriented at an angle of 90 degrees or more relative to a line extending outwardly away from the table assembly 101, parallel to the ground or to the lateral dimension 199 of the platform assembly 110, and perpendicular to the longitudinal dimension 197 of the rail 121 (i.e., a line that is generally perpendicular to a longitudinally extending side 109b of the platform 110). In other words, the nested configuration includes a configuration in which each of the proximal arms 134 is either oriented parallel with the longitudinal dimension 197 of the rail 121 and/or the longitudinal dimension 198 of the platform assembly 110 (see, for example, the proximal arm 134_2 in FIG. 3B), or extending in a direction toward a middle of the platform assembly 110 (see, for example, the proximal arm 134_1 in FIG. 3B). Moreover, in the nested configuration, the proximal arms 134 of the manipulators 140 are positioned adjacent to one another near an end portion of the platform assembly 110 (e.g., near a foot end portion in some embodiments and as shown in FIG. 3B) with the proximal arms 134 overlapping one another in at least one given direction. The nested configuration as described above allows the manipulators 140 to be moved fully or substantially fully away from obstructing the longitudinal extending side 109b of the platform assembly 110, which can be beneficial for various tasks that require or benefit from free space along the longitudinally extending side 109b, such a transferring a patient from a gurney to the platform assembly 110.

The angles of the proximal arms relative to the rail referred to herein are measured in the directions illustrated in the Figures, i.e., the angles are outward facing angles. In other words, if the sweep of the angles is traced starting from the rail, the angles initially sweep outwardly from the rail. Such outward facing angles are also referred to herein as outward angles. If the angles of the proximal arms are instead measured in the opposite direction from the rail, then the values referred to herein would be inverted (e.g., references to “greater than 180 degrees” would become “less than 180 degrees”, and vice versa).

In some embodiments, in the nested configuration the proximal arms 134 of the manipulators 140 overlap one another in a vertical direction, with one at a height higher than the other as measured from a ground surface, as shown in FIG. 3B. This can be referred to herein as a vertically nested configuration or vertical nesting. The vertically nested configuration can allow the more distal portions of the manipulators 140 to be moved fully around the end of the platform assembly 110 so as to be positioned along a laterally extending side 109a (end portion) of the platform assembly 110, as shown in FIGS. 1B, when it may be desired to move the manipulators 140 from a position along the longitudinally extending side 109b of the platform assembly 110, as illustrated in FIG. 1A. That is, distal portions of the manipulators 140 are moved in an arc around the end of the platform as the proximal arms 134 are rotated, such that the distal portions of the manipulators 140 move in a longitudinal direction (positive x-axis direction in FIG. 3B) beyond an end of the platform 110 and in a laterally inward direction (positive y-axis direction in FIG. 3B) past an outer edge of the longitudinally extending side 109b of the platform 110 to positions along the laterally extending side 109a of the platform 110. Thus, in some embodiments, the manipulators 140 can be fully moved out of the way of the longitudinally extending side 109b of the platform assembly 110 such that no portion of the manipulators 140 protrude laterally outward (e.g., in the negative y-axis direction in FIG. 3B) beyond an outer edge of the longitudinally extending side 109b of the platform assembly 110.

The vertically nested configuration illustrated in FIGS. 1B and 3B can be reached from the state of FIG. 1A by moving (translating) the manipulators 140 along the rail 121 toward a foot end of the platform assembly 110 and rotating the proximal arms 134 of the manipulators 140 until they are parallel or past parallel with the rail 121, i.e., to an outward angle q of 180 degrees or greater relative to the rail 121. For example, in FIG. 3B the proximal arms 134_1 and 134_2 are in the nested configuration and are oriented at outward angles φ_1 and φ_2 relative to the rail 121, respectively, with the outward angle φ_1 being greater than 180 degrees and the outward angle φ_2 being equal to 180 degrees. As the proximal arms 134 are rotated to this state, the more distal portions of the manipulators 140 swing around the end of the platform to their ultimate positions along the lateral extending side 109a. Thus, in the nested configuration the manipulators 140 are out of the way of and do not obstruct the longitudinally extending side 109b of the platform assembly 110, which can be desirable in various stages of a medical procedure, such as, for example, while transferring a patient from a gurney to the platform assembly 110. Moreover, in this position, the manipulators 140 can optionally remain in the sterile field and thus moving the manipulators 140 out of the way of the longitudinally extending side 109b of the platform assembly 110 for a task (e.g., patient transfer), can in some embodiments, not compromise the sterility of the manipulators 140. Accordingly, the manipulators 140 can be covered with a sterile drape prior to the task and remain sterile during the completion of that task without the need to otherwise move the manipulators 140 to a position outside the sterile field (e.g., in a stowed or semi-stowed state).

In some embodiments, the above-described nesting of the proximal arms 134 in the vertical direction is achieved by staggering the heights of the respective proximal arms 134 relative to the rail 121 such that a difference in height between the proximal arm 134 of one manipulator 140 (e.g., the proximal arm 134_1 in FIGS. 1A-D) and the proximal arm 134 of an adjacent manipulator 140 (e.g., the proximal arm 134_2 in FIGS. 1A-D) is sufficient to allow the adjacent proximal arms 134 to move over or under one another without collision. For example, as shown in FIGS. 1B and 2, a top surface of the proximal arm 134_1 is positioned a distance h₁ in a vertical direction below a bottom surface of the rail 121 and the proximal arm 134_2 is positioned a distance h₂ in the vertical direction below the rail 121, such that a difference in height Δh=h₂−h₁ is greater than the height dimension d of the proximal arm 134_1. As a result, the top surface of the proximal arm 134_2 is positioned lower than the bottom surface of the proximal arm 134_1, and thus the proximal arm 134_2 can move under the proximal arm 134_1 without collision. In some embodiments, this staggering in vertical position of the manipulators 140 is achieved by configuring the rail coupling portions 135 of the manipulators to have mutually different height dimensions. For example, in FIGS. 1A-3B the rail coupling portion 135_1 is part of the proximal arm 134_1 whereas the rail coupling portion 135_2 is a vertically extending component that extends downward from the rail 121 and couples to the proximal arm 134_2. In other words, the rail coupling portion 135_2 and the proximal arm 134_2 coupled thereto form an L-shaped portion, with one leg of the L-shaped portion (i.e., at least a part of the rail coupling portion 135_2) being oriented vertically and one leg (i.e., the proximal arm 134_2) being oriented horizontally in the nested configuration. This allows the proximal arm 134_2 to be positioned lower than the proximal arm 134_1. Although only two manipulators 140 are shown in the nested configuration in FIG. 1B, it should be understood that any number of manipulators 140 could be nested in this manner, for example by providing each successive manipulator 140 with a progressively longer rail coupling portion 135 and therefore a successively lower vertical positioning of the proximal arm 134 of the manipulator 140.

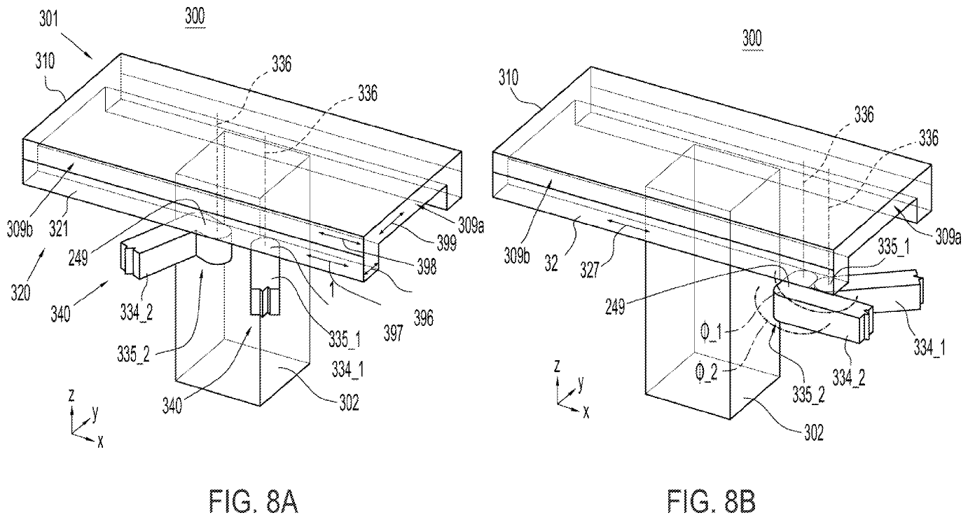

Although FIG. 1B shows the proximal arms 134 overlapping one another in the vertical dimension for ease of illustration, in some embodiments of the system 100 the proximal arms 134 overlap in a horizontal direction. In some of these embodiments, the coupling portion 135 and the proximal arm 134 of one manipulator 140 can form an L-shaped portion, with both legs of the L-shaped portion being oriented horizontally (i.e., within or parallel to the horizontal plane, described above) in the nested configuration. This allows the proximal arms 134 of all of the manipulators 140 coupled to the same rail 121 to be positioned adjacent to one another and horizontally overlapping while all are oriented at outward angles of 180 degrees or more relative to the rail 121. Such an embodiment of a system including proximal arms 134 overlapping in a horizontal direction is illustrated in FIGS. 8A and 8B and described further below. This may be referred to herein as a horizontally nested configuration or horizontal nesting. In embodiments in which the proximal arms horizontally nest, the manipulators might only be partially moved out of the way of the longitudinally extending side of the platform assembly, as portions of one of the manipulators can slightly protrude laterally beyond (outwardly from) the longitudinally extending side of the platform assembly.

In some embodiments, the proximal arm 134 includes a prismatic joint, which allows the proximal arm 134 to extend and retract. That is, in some embodiments the proximal arm 134 includes multiple links that can translate relative one another via the prismatic joint, thus extending or retracting the proximal arm 134. In some embodiments, the proximal arm 134 is coupled via a third rotational joint to an intermediate arm (including one or more links), which in turn can be coupled with additional more distal links via additional joints. In some embodiments, the first and/or second rotational joints of the rail coupling portion 135 and/or the prismatic joint of the proximal arm 134 are set-up joints, meaning that they are set during preparation for a procedure to establish a general position and pose of the manipulator 140, but then they are generally not moved, and in some embodiments locked mechanically and/or via software, during the performance of the procedure under the control of the user, whereas more distal joints of the manipulator 140 can be movable throughout the procedure in response to user inputs.

In some embodiments, the rail coupling portion 135 of at least one manipulator 140 further includes a second rotational joint (not illustrated) configured to rotate the attached proximal arm 134 around a second axis 137 (see FIGS. 1A and 4A-4D) parallel to the longitudinal dimension 197 of the rail 121. This rotation allows the proximal arm 134 to be inclined or declined relative to the horizontal plane. The horizontal plane is a plane parallel to the lateral dimension 196 of the rail 121 (see FIG. 2) and longitudinal dimension 197 of the rail 121 (see FIG. 1A). In some embodiments (e.g., embodiments in which the rail 121 is attached to the platform 110), the horizontal plane is also parallel to the longitudinal and lateral dimensions 198 and 199 of the platform assembly 110. In other embodiments (e.g., embodiments in which the rail 121 is attached to the support column 102), the horizontal plane can be parallel to the longitudinal and lateral dimensions 198 and 199 of the platform assembly 110 in a neutral state of the platform assembly 110, but not necessarily in other states. In some embodiments (e.g., embodiments in which the rail 121 is coupled to the support column 102), the horizontal plane is also parallel to the ground or other supporting surface. In other embodiments (e.g., embodiments in which the rail 121 is coupled to the platform 110), the horizontal plane is parallel to the ground or other supporting surface in a neutral state of the platform 110, but not necessarily in other states. Inclining the proximal arm 134 can increase the reach of the manipulator 140, for example allowing a distal end portion, including an instrument mount, of the manipulator 140 to reach over the platform assembly 110 (and over a patient supported thereon) to a position on an opposite longitudinal side of the platform assembly 110 than the rail assembly 120. This can allow a manipulator 140 attached to one side of the platform assembly 110 to nevertheless reach places that would normally need a manipulator 140 attached to the other side of the platform assembly 110. This can be beneficial when more manipulators 140 are needed on one side of the platform assembly 110 than are available, including, for example, in embodiments in which manipulators 140 are provided on only one side of the platform assembly 110.

FIGS. 4A-4D schematically illustrate an embodiment of a system 160, which can be used as the system 100. The system 160 includes many of the same parts as the system 100, and thus the same reference numbers are used for these parts and duplicative descriptions thereof are omitted. The system 160 includes manipulators 140 including proximal arms 134 and coupling portions 135, as described above (only a proximal portion of the proximal arm 134 of each manipulator is illustrated in FIGS. 4A-4D). In the system 160, one of the manipulators 140 includes a coupling portion 135_2 that has two rotational joints, specifically a first rotational joint (not visible in the exterior view of the figures) to provide rotation of the proximal arm 134_2 around a first axis 136_2 perpendicular to the longitudinal dimension 197 of the rail 121 and a second rotational joint (not visible in the exterior view of the figures) to provide rotation of the proximal arm 134_2 around a second axis 137 aligned with the longitudinal dimension 197 of the rail 121. FIG. 4A illustrates the coupling portion 135_2 and proximal arm 134_2 in a neutral state in which the proximal arm 134_2 is horizontal (i.e., parallel to a horizontal plane defined by the lateral and longitudinal dimensions 196 and 197 of the rail 121), FIG. 4B illustrates the coupling portion 135_2 and proximal arm 134_2 in a partially inclined state in which the proximal arm 134_2 has been rotated around the axis 137 such that the proximal arm 134_2 is inclined relative to the horizontal plane. FIG. 4C illustrates the coupling portion 135_2 and proximal arm 134_2 in a fully inclined state in which the proximal arm 134_2 has been rotated around the second axis 137 to the point that proximal arm 134_2 is perpendicular to the horizontal plane. FIG. 4D illustrates the coupling portions 135_1 and 135_2 and proximal arms 134_1 and 134_2 in the nested configuration at one end portion of the platform assembly 110. As shown in FIG. 4A, in the neutral position of the manipulator 140 in which the proximal arm 134_2 is horizontal (extending in a direction generally perpendicular and away from of the longitudinal side 109b of the platform assembly 110), the first axis 136_2 about which the proximal arm 134_2 rotates relative to the coupling portion 135_2 is vertical (perpendicular to the horizontal plane defined by the lateral and longitudinal dimensions 196 and 197 of the rail 121), but as the proximal arm 134_2 rotates around the second axis 137, the first axis 136_2 changes orientation. As shown in FIGS. 4B and 4C, inclining the proximal arm 134_2 by rotation around the second axis 137 increases a height of the distal end portion of the proximal arm 134_2, thus allowing the more distal portions of the manipulator 140 that are coupled to the proximal arm 134_2 to have a greater reach.

As shown in FIGS. 4A-4D, in some embodiments the coupling portions 135_2 that includes the first and second rotational joints can include a first part 138 and a second part 139. The first and second joints can be housed within the first and second parts 138 and 139. In some embodiments, the first part 138 of the coupling portion 135_2 is movably coupled to the rail 121 via a first carriage 126 (not illustrated in FIGS. 4A-4D, reference made to FIGS. 1A-1B), which can be coupled to, or can be part of, the first part 138. In some embodiments, the first part 138 is rotatably coupled to the second part 139 via the aforementioned second rotational joint, such that the second part 139 can rotate around the second axis 137 relative to the first part 138, as shown in FIGS. 4A-4C. In some embodiments, the second part 139 is rotatably coupled to the proximal arm 134_2 via the first rotational joint such that the proximal arm 134_2 can rotate around the first axis 136_2 relative to the second part 139. In some embodiments, the above-described first and/or second rotational joints of the rail coupling portion 135_2 are unpowered joints. In other embodiments, the first and/or second rotational joints of the rail coupling portion 135_2 are powered joints driven by actuators (such as motors or other actuators familiar to those having ordinary skill in the art), which can be positioned inside the rail coupling portion 135 and/or in the proximal arm 134. The above-described arrangement of the first and second parts 138 and 139 is merely one embodiment, and other arrangements are contemplated and would be appreciated by those of ordinary skill in the art based on the present disclosure. For example, in some embodiments the first part 138 is rotatable relative to the rail 121 around a vertical axis, instead of or in addition to the second part 139 being rotatably coupled to the proximal arm 134_2. In addition, in some embodiments the coupling portion 135_2 can include more or fewer parts in addition to or instead of the first and second parts 138 and 139. Any arrangement or parts and joints can be used that provides for at least the ability for the proximal arm 134_2 to rotate around a second axis 137 and a first axis 136 perpendicular to the second axis 137.

The configuration of the manipulators 140 that allows them to be nested and thus moved out of the way of the longitudinal side 109b of the platform 110 can also provide additional benefits. For example, the structures that provide for the nesting of the proximal arms 134 can also facilitate an easier and/or more compact stowing of the manipulators 140. In embodiments in which the proximal arms 134 can be vertically nested in a deployed state, the same structural configuration can also allow the proximal arms 134 to be vertically nested in a stowed state (see FIG. 5 as one embodiment), which can allow for more compact stowing of the manipulators 140 below the platform assembly 110. In other words, in such a stowed state the proximal arms 134 of those manipulators 140 can be oriented at outward angles 180 degrees or more relative to the rail 121 (i.e., parallel to the rail 121 or angled to extend in an inward direction toward a middle of the platform 110) and can vertically overlap (i.e., the proximal arms 134 are vertically stacked atop one another). Similarly, in embodiments in which the proximal arms 134 can be horizontally nested in a deployed state, the same structural configuration can also allow the proximal arms 134 to be horizontally nested in a stowed state (not illustrated).

Returning to FIGS. 1A-2 and the system 100, the instrument 150 can be removably mounted to the manipulator 140 via an interface or can be permanently mounted to the manipulator 140. The instruments 150 can include any tool or instrument, including for example industrial instruments and medical instruments (e.g., surgical instruments, imaging instruments, diagnostic instruments, therapeutic instruments, etc.). In embodiments in which the instrument 150 is removably mountable to the manipulator 140, the manipulator 140 can include an instrument manipulator mount (not illustrated) to which the instrument can be removably coupled. The instrument manipulator mount can be located, for example, at a generally distal end portion of the manipulator 140, and has an interface (not illustrated) including output couplers (not illustrated) to engage (directly or indirectly via an intermediary) with input couplers (not illustrated) of the instrument 150 to provide driving forces or other inputs to the mounted instrument 150 to control various degree of freedom movement and/or other functionality of the instrument 150, such as moving an end-effector of the instrument, opening/closing jaws, driving translation and/or rotation of a variety of components of the instrument, delivery of substances and/or energy from the instrument, and various other functions those of ordinary skill in the art are familiar with. The output couplers can be driven by actuators (e.g., electrical servo-motors, hydraulic actuators, pneumatic actuators) with which those of ordinary skill in the art have familiarity. An instrument sterile adaptor (ISA) can be disposed between the instrument 150 and the instrument manipulator mount interface to maintain sterile separation between the instrument 150 and the manipulator 140. The instrument manipulator mount can also include other interfaces (not illustrated), such as electrical interfaces to provide and/or receive electrical signals to/from the instrument 150. In some embodiments, the system 100 can include flux delivery transmission capability as well, such as, for example, to supply electricity, fluid, vacuum pressure, light, electromagnetic radiation, etc. to the end effector. In other embodiments, such flux delivery transmission can be provided to an instrument through another auxiliary system 1008, described further below and as those of ordinary skill in the art would be familiar with in the context of computer-assisted, teleoperated medical systems.

In some embodiments, the manipulators 140 can be similar to the manipulators described in U.S. Provisional Patent Application No. 63/336,773, entitled “RAIL ASSEMBLY FOR TABLE MOUNTED MANIPULATOR SYSTEM, AND RELATED DEVICES, SYSTEMS AND METHODS,” inventor Ryan Abbott, and in U.S. Provisional Patent Application No. 63/336,840, entitled “TABLE-MOUNTED MANIPULATOR SYSTEM, AND RELATED DEVICES, SYSTEMS AND METHODS,” first named inventor Steven Manuel, both of which were filed on Apr. 29, 2022, or those described in, for example, U.S. Pat. No. 9,358,074 (filed May 31, 2013) to Schena et al., entitled “MULTI-PORT SURGICAL ROBOTIC SYSTEM ARCHITECTURE,” U.S. Pat. No. 9,295,524 (filed May 31, 2013) to Schena et al., entitled “REDUNDANT AXIS AND DEGREE OF FREEDOM FOR HARDWARE-CONSTRAINED REMOTE CENTER ROBOTIC MANIPULATOR,” and U.S. Pat. No. 8,852,208 (filed Aug. 12, 2010) to Gomez et al., entitled “SURGICAL SYSTEM INSTRUMENT MOUNTING,” the contents of all of which are incorporated herein by reference in their entirety. Various other embodiments of manipulators can include those as configured as part of the medical systems that are part of various da Vinci® Surgical Systems, such as the da Vinci X®, da Vinci Xi®, and da Vinci SP systems, commercialized by Intuitive Surgical, Inc., of Sunnyvale, California.

As shown in FIGS. 1A-2, the manipulators 140 are coupled to the table assembly 101 via the at least one rail assembly 120. In some embodiments, multiple similar rail assemblies 120 are provided, for example one for each longitudinal side of the platform assembly 110. For example, in some embodiments, a first rail assembly 120 can be provided at a first longitudinal side of the platform assembly 110 and a second rail assembly 120 can be provided at a second longitudinal side of the platform assembly 110. In such embodiments with multiple rail assemblies 120, manipulators 140 can be coupled the rail assemblies 120 in any number or combination, and because the rail assemblies 120 can be positioned along multiple sides of the platform assembly 120, the manipulators 140 too can be positioned along multiple sides of the platform assembly 140. The description below will describe one rail assembly 120 to simplify the description, but the other rail assemblies 120 (if present) can be configured similarly. The rail assembly 120 includes a rail 121 and two or more first carriages 126 (two being shown in the embodiment of FIGS. 1A-2) coupled to the rail 121 and to the manipulators 140 to allow motion of the manipulators 140 along the rail 21. More specifically, the first carriage 126 can be coupled to (or can be a part of) the rail coupling portion 135 of a manipulator 140. Each first carriage 126 is moveable along a longitudinal dimension 197 of the rail 121 and couples a respectively corresponding one of the manipulators 140 to the rail 121 such that the manipulators 140 can translate relative to the rail 121 along the longitudinal dimension 197 of the rail 121. In some embodiments, the longitudinal dimension 197 of the rail 121 is parallel to the longitudinal dimension 198 of the platform assembly 110 (e.g., parallel to the x-axis) in a neutral configuration of the platform assembly 110, as shown in FIG. 1A.

The rail 121 includes a first set of engagement features 122 configured to engage with complementary engagement features 128 of the first carriages 126. For example, as shown in FIG. 2, the first set of engagement features 122 can include a track including flanges extending along the longitudinal dimension 197, and the complementary engagement features 128 of the first carriages 126 are configured to engage and ride along the flanges of the first set of engagement features 122. The first set of engagement features 122 can also include a track including grooves in which the complementary engagement feature 128 are received. Any other type of complementary engagement features that when engaged allow for relative translation can be used as the complementary engagement features 128, and those having ordinary skill in the art are familiar with various complementary engagement features that are used in rail and carriage systems. In some embodiments, the first set of engagement features 122 and/or the complementary engagement features 128 can include bearing devices configured to reduce friction to facilitate easier translation, such as wheels, balls, plain bearing surfaces coated or otherwise provided with a low friction material, and other friction reducing mechanisms. In FIGS. 1A-2, one first carriage 126 is shown per manipulator 140, but multiple first carriages 126 could be provided to operably couple to and support a given manipulator 140.

In some embodiments, in addition to the manipulators 140 being movable (in translation) along the rail 121, the rail 121 can also be movable relative to the table assembly 101. In such embodiments, the rail assembly 120 also includes one or more second carriages 127 coupled to the rail 121 and to the table assembly 101 to allow motion of the rail 121. More specifically, the one or more second carriages 127 couple the rail 121 to the table assembly 101 such that the rail 121 can translate relative to the table assembly 101 along a direction of the longitudinal dimension 197 of the rail 121. In particular, as shown in FIG. 2, the rail 121 includes a second set of engagement features 123 (e.g., tracks or other engagement features) that engage with complementary engagement features 129 of the second carriage 127 to couple the rail 121 to the second carriage 127 while allowing translation between the rail 121 and second carriage 127. The engagement features 123 and complementary engagement features 129 can be similar to the engagement features 1212 and complementary engagement features 128 described above.

In some embodiments, the translation between the rail 121 and the table assembly 101 is provided solely by relative motion between the second carriages 127 and the rail 121. For example, in some embodiments the second carriages 127 are fixed relative to the table assembly 101 and the rail 121 and second carriages 127 are movably coupled together such that the rail 121 translates relative to the second carriages 127 along the direction of the longitudinal dimension 197 of the rail 121. In other embodiments, the translation of between the rail 121 and the table assembly 101 is provided by relative motion between the second carriages 127 and the table assembly 101. For example, in some embodiments the second carriages 127 are fixed relative to the rail 121 and movably coupled to the table assembly 101 such that translation of the second carriages 127 relative to the table assembly 101 along the longitudinal dimension 197 causes the rail 121 to also translate relative to the table assembly 101. In some embodiments, the translation between the rail 121 and the table assembly 101 is provided by a combination of relative motion between the second carriages 127 and the rail 121 and relative motion of the second carriages 127 and the table assembly 101. In some embodiments in which the second carriages 127 are movably coupled to the table assembly 101, the rail assembly 120 further includes a second rail 124, which can be coupled between the second carriages 127 and the table assembly 101. For example, the second rail 124 can include engagement features 125 to engage the second carriages 127 such that the second carriages 127 can translate along the rail 124. In other embodiments, the second rail 124 is omitted. One second carriage 127 is shown in FIGS. 1A-2 for ease of description, but any number could be used.

The movability of the rail 121 relative to the table assembly 101 (in embodiments in which such motion is provided) can allow for a greater range of motion of the manipulators 140 and/or for a shortening of the rail 121, as compared to a configuration in which the rail is fixed relative to the table assembly 101. This can also enable the rail assembly 120 to more easily be moved out of the way of the platform assembly 110 to avoid interference therewith as the platform assembly 110 and/or individual platform sections 103 thereof are moved through various configurations. However, in some embodiments the rail 121 is fixed relative to the table assembly 101 and the manipulators 140 are positioned relative to the platform assembly 110 solely through motion of the manipulators 140 along the rail 121.

In some embodiments, the rail assembly 120 is coupled to one of the platform sections 103. In other embodiments, the rail assembly 120 is coupled to the support column 102. Which structure the rail assembly 120 is coupled to can make a difference in embodiments in which the platform assembly 110 as a whole is movable relative to the support column 102, for example by tilting relative to the support column 102. In embodiments in which the rail assembly 120 is coupled to one of the platform sections 103 (e.g., a middle section 103_2), when the platform assembly 110 moves relative to the support column 102, the rail assembly 120 and hence the manipulators 140 coupled thereto move along with the platform assembly 110. This can allow the manipulators 140 to automatically maintain a set pose and position relative to the platform assembly 110, and thus relative to a patient supported on the platform assembly, regardless of a configuration of the platform assembly 110 and without having to reposition the manipulators 140. Moreover, in some circumstances, collision between the platform assembly 110 and the rail assembly 120 due to motion of the platform assembly 110 can be avoided as they both move together. In embodiments in which the rail assembly 120 is coupled to the support column 102, when the platform assembly 110 moves relative to the support column 102, the rail assembly 120 and hence the manipulators 140 coupled thereto remain with the support column 102 and do not move along with the platform assembly 110. This can allow for greater stiffness of the rail assembly 120 relative to the base 105 (since there are fewer elements in the kinematic chain), and/or can allow for an increased relative range of motion between manipulators 140 and the table assembly 110 (since the table assembly 110 can be movable relative to the rail 121 along additional degrees of freedom of motion)].

In some embodiments, motors or other actuation devices (not illustrated but with which those of ordinary skill in the art have familiarity) are provided to drive the relative translation between the rail 121 and the first carriages 126. Similarly, in embodiments in which the second carriages 127 are present, motors or other actuation devices (not illustrated) can be provided to drive the relative translation between the rail 121 and the second carriages 127 and/or between the second carriages 127 and the table assembly 101. In some embodiments, motors/actuators are housed within the rail 121. In some embodiments, motors/actuators are housed within the first and/or second carriages 126 and 127.

The motion of the manipulators 140 relative to the platform assembly 110 enabled by the rail assembly 120 allows the coupling portions 135 of the manipulators 140 to be moved adjacent to one another and to near an end portion of the platform assembly 110 (e.g., near an end portion of the rail 121), and when the coupling portions 135 are so positioned and the manipulators 140 are placed in the nested configuration described above, the distal portions of the manipulators 140 are beyond an end of the platform assembly 110 and positioned adjacent a lateral side 109a of the platform assembly 110 while deployed. In the embodiments of FIGS. 1A-4D the manipulators 140 are configured to be moved beyond the foot end of the platform assembly 110 in the nested configuration while deployed. In other embodiments, the manipulators 140 can be moved beyond the head end of the platform assembly 110, and in still other embodiments the manipulators 140 can be moved beyond both the head end and beyond the foot end, respectively.

Returning to FIG. 1A, the user input and feedback system 1004, control system 1006, and auxiliary system 1008 will be described. Some or all of these components can be provided at a location remote from the table assembly 101. The user input and feedback system 1004 is operably coupled to the control system 1006 and includes one or more input devices to receive input control commands to control operations of the manipulators 140, instruments 150, rails assembly 120, and/or table assembly 101. Such input devices can include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device can be provided with the same degrees of freedom as the associated instrument that they control, and as the input device is actuated, the instrument, through drive inputs from the manipulator assembly, is controlled to follow or mimic the movement of the input device, which can provide the user a sense of directly controlling the instrument. Telepresence input devices can provide the operator with telepresence, meaning the perception that the input devices are integral with the instrument. The user input and feedback system 1004 can also include feedback devices, such as a display device (not shown) to display images (e.g., images of the workspace as captured by one of the instruments 1010), haptic feedback devices, audio feedback devices, other graphical user interface forms of feedback, etc.

The control system 1006 can control operations of the system 100. In particular, the control system 1006 can send control signals (e.g., electrical signals) to the table assembly 101, rail assembly 120, manipulators 140, and/or instruments 150 to control movements and/or other operations of the various parts. In some embodiments, the control system 1006 can also control some or all operations of the user input and feedback system 1004, the auxiliary system 1008, or other parts of the system 100. The control system 1006 can include an electronic controller to control and/or assist a user in controlling operations of the manipulator assembly 1001. The electronic controller includes processing circuitry configured with logic for performing the various operations. The logic of the processing circuitry can include dedicated hardware to perform various operations, software (machine readable and/or processor executable instructions) to perform various operations, or any combination thereof. In examples in which the logic includes software, the processing circuitry can include a processor to execute the software instructions and a memory device that stores the software. The processor can include one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In cases in which the processing circuitry includes dedicated hardware, in addition to or in lieu of the processor, the dedicated hardware can include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry can also include any combination of dedicated hardware and processor plus software.

Differing degrees of user control versus autonomous control can be utilized in the system 100, and embodiments disclosed herein can encompass fully user-controlled systems, fully autonomously-controlled systems, and systems having any combination of user and autonomous control. For operations that are user-controlled, the control system 1006 generates control signals in response to receiving a corresponding user input command via the user input and feedback system 1004. For operations that are autonomously controlled, the control system 1006 can execute pre-programmed logic (e.g., a software program) and can determine and send control commands based on the programming (e.g., in response to a detected state or stimulus specified in the programming). In some systems, some operations can be user controlled and others autonomously controlled. Moreover, some operations can be partially user controlled and partially autonomously controlled—for example, a user input command can initiate performance of a sequence of events, and then the control system 1006 can perform various operations associated with that sequence without needing further user input.

In some embodiments, the control system 1006 can control positions of the manipulators 140 and the positions or configurations of the rail assembly 120 and/or the table assembly 101 by sending electrical signals to drive actuators that move the manipulators 140, the rail assembly 120, and/or the table assembly 101. In some embodiments, the control system 1006 can determine that a predetermined task is to be performed, such as a transfer of a patient to the table assembly 101, and in response can automatically drive the manipulators 140 and/or the rail assembly 120 to move the coupling portions 135 of the manipulators 140 to one end of the platform assembly 110 and then drive the manipulators 140 to move into the nested configuration. Driving the manipulators 140 to move into the nested configuration can include driving the one or more rotary joints in the coupling portion 135 to cause the proximal arms 134 of the manipulators 140 to rotate to outward angles at least 180 degrees relative to the rail 121, as well as adjusting other joints of the manipulators 140 to obtain a desired pose of the manipulators 140. For example, the desired pose can be a pose in which the manipulators 140 are positioned fully out of the way of the longitudinal side 109b of the platform assembly 110. The control system 1006 can determine that the predetermined task is going to occur by, for example, detecting one or more sensor signals (e.g., sensing proximity of a gurney to the table assembly 101 by sensing wireless signals emitted by a device on the gurney) and/or or by receiving inputs, commands, or other signals from a user interface indicative that the task is to be performed (e.g., a user pressing a button associated with beginning the task).

The auxiliary system 1008 can include various auxiliary devices that can be used in operation of the system 100. For example, the auxiliary system 1008 can include power supply units, auxiliary function units (e.g., functions such as irrigation, evacuation, energy supply, illumination, sensors, imaging, etc.). As one example, in a system 100 for use in a medical procedure context, the auxiliary system 1008 can include a display device for use by medical staff assisting a procedure, while the user operating the input devices can utilize a separate display device that is part of the user input and feedback system 1004. As another example, in a system 100 for use in a medical context, the auxiliary system 1008 can include flux supply units that provide surgical flux (e.g., electrical power) to instruments 150. An auxiliary system 1008 as used herein can thus encompass a variety of components and does not need to be provided as an integral unit.

Turning now to FIGS. 5-7D, another embodiment of a table-mounted manipulator system 200 (“system 200”) is described below. The system 200 can be used as the system 100, and some components of the system 200 can be used as components of the system 100 described above. Thus, the descriptions of the components of the system 100 above are applicable to the related components of the system 200, and duplicative descriptions of these components are omitted below. The related components of the systems 100 and 200 are given reference numbers having the same right-most two digits—for example, 105 and 205. Although the system 200 is one embodiment of the system 100, the system 100 is not limited to the system 200.

As shown in FIGS. 5-7D, the system 200 includes a table assembly 201, two rail assemblies 220 coupled to the table assembly on opposite longitudinal sides, and multiple manipulators 240 coupled to the rail assemblies 220. Each manipulator 240 is configured to carry one more instruments (not illustrated), which can be removably or permanently mounted thereon. The system 200 also can include a control system (not illustrated), a user input and feedback system (not illustrated), and/or an auxiliary system (not illustrated) similar to those described above in relation to the system 100. In some embodiments, the system 200 is configured as a computer-assisted, teleoperable medical system. In other embodiments, the system 200 is configured as a teleoperable system for use in non-medical contexts.

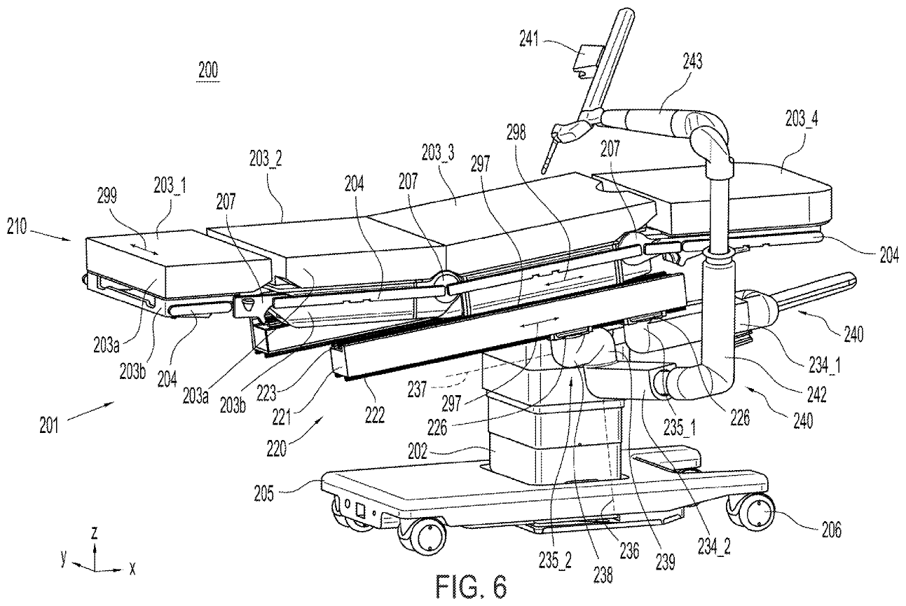

As shown in FIGS. 5 and 6, the table assembly 201 includes a platform assembly 210 (also “platform 210”) configured to support the patient or inanimate workpiece, a support column 202 coupled to and supporting the platform assembly 210, and a base 205 coupled to the support column 202. The base 205 can be configured to contact the ground or other surface upon which the table assembly 201 rests, and in some embodiments the base 205 includes wheels 206 to allow movement of the system 200 along the ground or other surface. As shown in FIG. 6, in some embodiments the support column 202 includes a telescoping support column that can raise or lower the platform assembly 210.

The platform assembly 210 includes multiple platform sections 203 configured to support the patient or workpiece. In particular, in the embodiment illustrated in FIGS. 5-7D the platform assembly 210 includes first end section 203_1 (“head section 203_1”), middle sections 203_2 and 203_3, and second end section 203_4 (“foot section 203_4”), which are arranged in series and movably coupled together via joints 207. In some embodiments, the first end section 203_1 can be configured to support a head of the patient, the second end section 203_4 can be configured to support the feet and/or legs of the patient, and the more middle sections 203_2 and 203_3 can be configured to support a torso and/or other portions of the patient. The joints 207 allow adjacent platform sections 203 to pivot relative to one another about rotation axes parallel to a lateral dimension 299 of the platform assembly 210 (e.g., parallel to a y-axis in the Figures). FIG. 5 illustrates the platform assembly 210 in a neutral configuration in which all of the platform sections 203 are parallel to one another, and FIG. 6 illustrates the platform assembly 210 in an articulated configuration in which some of the platform sections 203 are oriented at non-zero angles relative to adjacent platform sections 203. In some embodiments, some of the joints 207 can also allow for other motion between adjacent platform sections 203, such as relative translation along the longitudinal dimension 298 or relative rotation around a vertical axis parallel to a height dimension (i.e., the z-axis in the Figures), which is perpendicular to the lateral and longitudinal dimensions 299 and 298. In some embodiments, the platform sections 203 include harder support portions 203b and softer cushion portions 203a attached to the support portions 203b, with a surface of the cushion portions 203a (i.e., the top surface in the orientation illustrated in FIGS. 5 and 6) forming a support surface that contacts the patient or workpiece. In some embodiments, multiple platform sections 203 can share some components. For example, as illustrated in FIG. 5, the middle platform sections 203_2 and 203_3 can share the same cushion portion 203a that extends across both platform sections 203_2 and 203_3. The cushion portion 203a shared by the platform sections 203_2 and 203_3 can bend when the platform sections 203_2 and 203_3 are articulated relative to one another, as shown in FIG. 6.

In addition to moving individual platform section 203 relative to adjoining platform sections 203, the platform assembly 210 as a whole is movable relative to the support column 202. In some embodiments, the middle section 203_3 is coupled to the support column 202 by one or more joints (not illustrated), providing for motion between the middle section 203_3 and the column 202. The other platform sections 203_1, 203_2, and 203_4 are coupled (directly or indirectly) to the middle section 203_3, and thus as the middle section 203_3 moves relative to the support column 202 the platform assembly 210 as a whole moves relative to the support column 202. In some embodiments, the motion of the middle section 203_3 (and hence platform assembly 210 as a whole) relative to the support column 202 includes pivoting (tilting) about a horizontal axis parallel to the lateral dimension 299 (e.g., a pitch degree of freedom of motion), as shown in FIG. 6. In some embodiments, other degrees of freedom of motion are provided between the middle section 203_3 and the support column 202, including pivoting (tilting) about a horizontal axis parallel to the longitudinal dimension 298 (e.g., a roll degree of freedom of motion), rotating about a vertical axis (e.g., a yaw degree of freedom of motion), and/or translation along the lateral and/or longitudinal dimensions 299 or 298. As shown in FIG. 6, in the neutral configuration of the platform assembly 210, the platform assembly 210 is parallel to the ground (or other surface upon which the system 200 rests)—in particular, the support surfaces of the platform sections 203 are parallel to the ground (or other surface).

As shown in FIGS. 5-7D, the platform assembly 210 also includes a number (one or more) of accessory rails 204 attached to side surfaces of the support portions 203b of the platform sections 203.

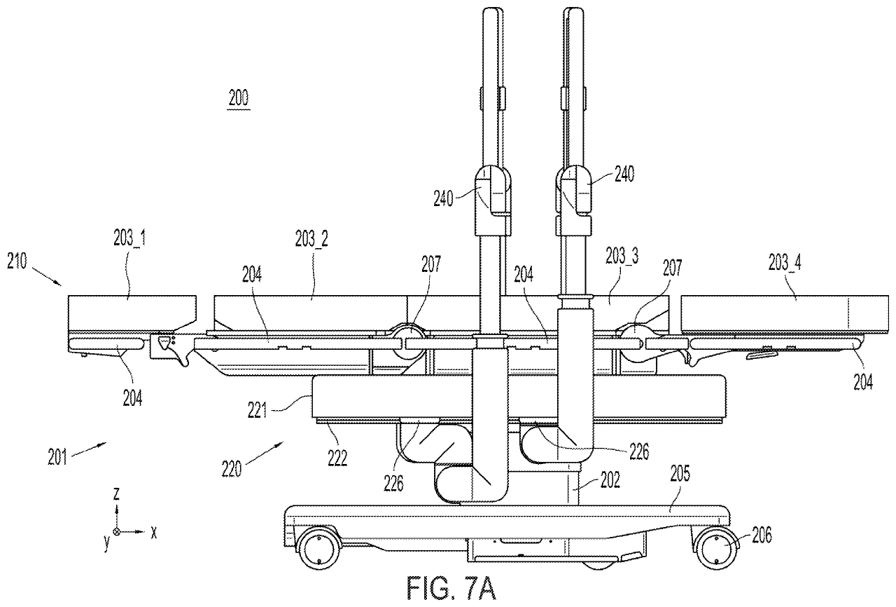

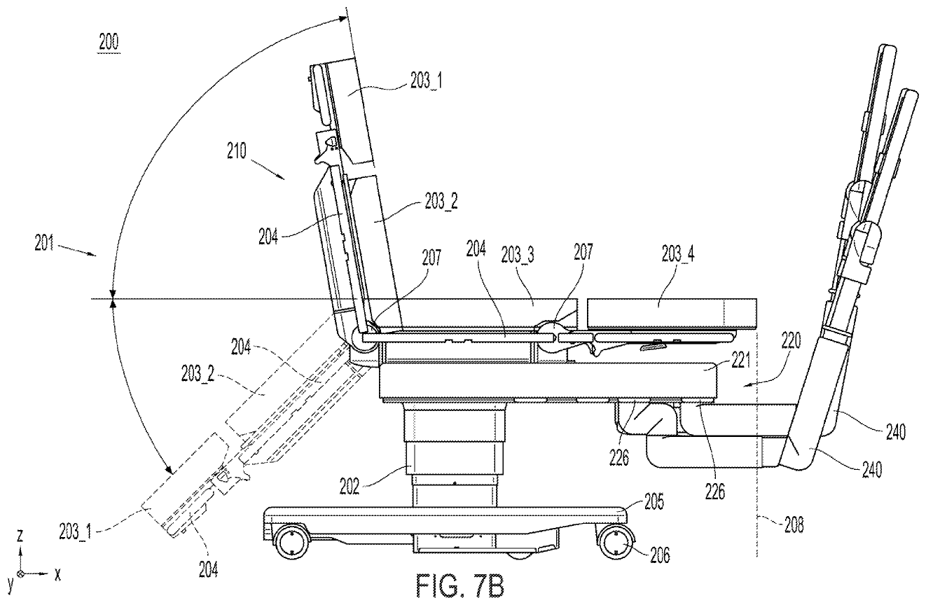

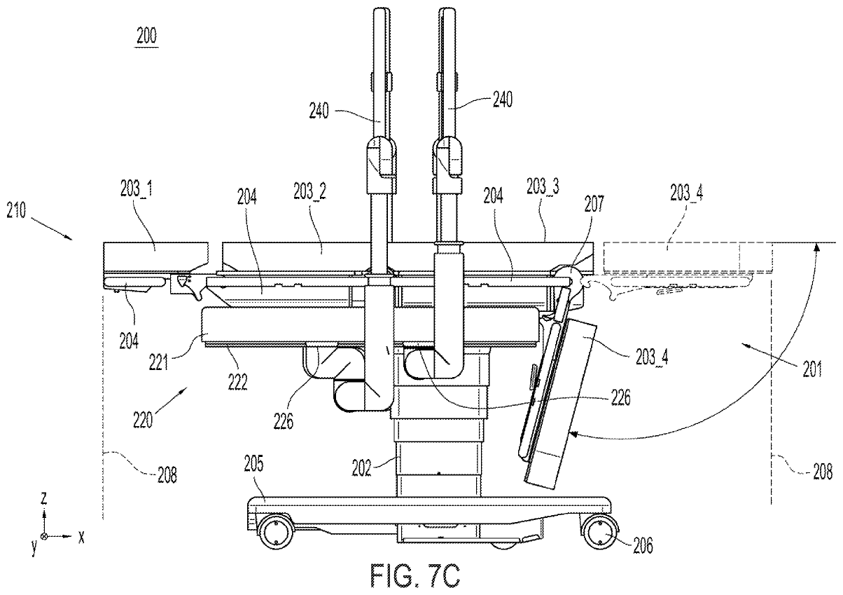

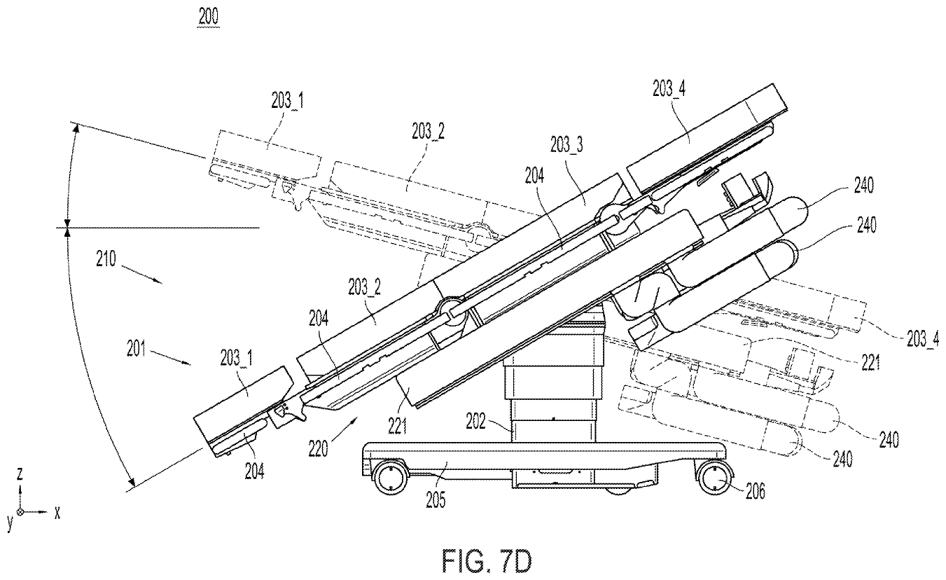

As noted above, the system 200 includes multiple manipulators 240. In the embodiment illustrated in FIG. 5, four manipulators 240 are present, with two manipulators 240 on each longitudinally extending side of the platform assembly 210. In other embodiments, more or fewer manipulators 240 can be used. In FIGS. 5 and 7D, the manipulators 240 are shown in a stowed state under end section 203_4, while in FIGS. 6-7C the manipulators 240 are shown in various deployed states (only the two manipulators 240 disposed on one side of the platform assembly 210 are visible in FIGS. 6-7D, and only one of the manipulators 240 is in a deployed state in FIG. 6). The deployed states include states in which the manipulators 240 are not stowed, e.g., distal ends of the manipulators 240 are positioned at or above the level of the platform assembly 110, and can include a variety of configurations and positions of the manipulators 240 including but not limited to those shown in FIGS. 6-7C.

As shown in FIGS. 5-7D, the manipulators 240 are coupled to the table assembly 201 via the rail assemblies 220. One rail assembly 220 is provided for each of the two longitudinally extending sides of the platform assembly 210. The description below describes a single rail assembly 220 for ease of description, and the other rail assembly 220 can be configured similarly. As shown in FIGS. 5 and 6, the rail assembly 220 includes a movable rail 221 (“rail 221”), first carriages 226 coupled to the rail 221 and to the manipulators 240 (at least one first carriage 226 per manipulator 240), and one or more second carriages 227 coupled to the rail 221 and the table assembly 201 (see FIG. 6). In the embodiment illustrated in FIGS. 5-7D, the rail 221 is movably coupled to the platform 210 via engagement features 223 such that the rail 221 translates along the longitudinal dimension 298 of the platform 210 and moves along with the platform 210 relative to the support column 202.

Each first carriage 226 couples a respectively corresponding one of the manipulators 240 to the rail 221 such that the manipulators 250 can translate relative to the rail 221 along a longitudinal dimension 297 of the rail 221. In particular, each first carriage 226 includes complementary engagement features coupled to engagement features 222 of the rail 221. Each first carriage 226 is coupled to (or is part of) a coupling portion 235 of a corresponding manipulator 240. The coupling portions 235 are coupled to (or are part of) corresponding proximal arms 234. Each proximal arm 234 includes one or more links, and is coupled to more distal portions of the manipulator 240, which can include, for example, an intermediate arm 242, a distal arm 243, an instrument manipulator mount 241 configured to receive an instrument removably mounted thereon, and a number of joints 245 movably coupling the various arms of the distal portions together. The instrument manipulator mount 241 includes an interface with output couplers to transfer driving force or other inputs to the instrument. In some embodiments, the proximal arm 234 and/or the intermediate arm 242 are telescoping arms, in which case the proximal arm 234 and/or the intermediate arm 242 can each include multiple links that are translatable relative to on another.

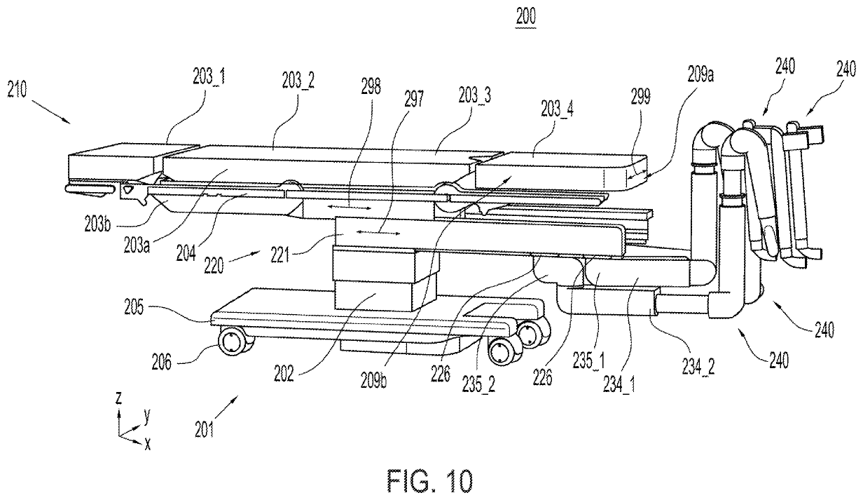

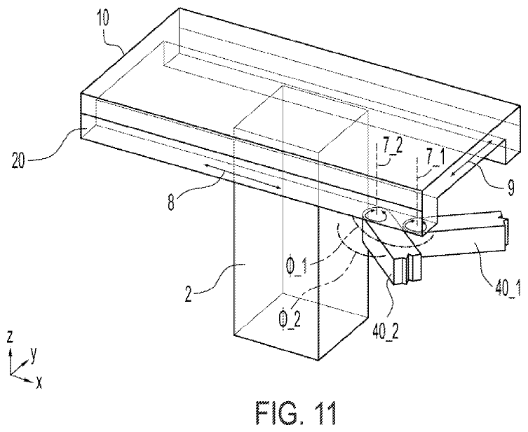

As shown in FIG. 6, the coupling portion 235_2 of one manipulator 240 is longer in a height dimension than the coupling portion 235_1 of the other manipulator 240 coupled to the same rail 221, and as a result the proximal arm 234_2 coupled to the coupling portion 235_2 is positioned lower in a vertical direction (z-axis direction in the Figures) than the proximal arm 234_1. The difference in height between the proximal arm 234_1 and the proximal arm 234_2 is sufficiently large that the proximal arm 234_2 can be moved under the proximal arm 234_1 without collision, allowing the manipulators 240 to be placed in a vertically nested configuration similar to the vertically nested configuration described above in relation to the system 100. For example, FIGS. 7B, 9, and 10 illustrate the manipulators 240 in a deployed and nested configuration, and FIG. 7D illustrates the manipulators 240 in a stowed and nested configuration. As described above and as shown in FIGS. 7B, 9, and 10, in the nested configuration the proximal arms 234 of the manipulators 240 coupled to the same rail 221 are oriented at outward angles of 180 degrees or more relative to the longitudinal dimension 297 of the rail 221 and overlap one another vertically (i.e., in a direction perpendicular to the lateral and longitudinal dimensions 296 and 297 of the rail 221). For example, in FIG. 9 the proximal arm 234_1 is at an outward angle φ_1 that is greater than 180 degrees and the arm 243_2 is at an outward angle φ_2 that is equal to 180 degrees relative to the rail 221. When the manipulators are in a deployed state and in the nested configuration, as shown in FIGS. 7B, 9, and 10, the distal portions of the manipulators (e.g., the intermediate arm 242, distal arm 243, and instrument holder 241) are swung around an end of the platform 210 to positions along a laterally extending side 209a and instead of positions along a longitudinally extending side 209b.

As shown in FIG. 6, the coupling portion 235_2 includes a first part 238 and a second part 239, similar to the coupling portion 135_2 illustrated in FIGS. 4A-4D. The proximal arm 234_2 is rotatably coupled to the second part 239 via a first rotary joint (not visible) such that the proximal arm 234_2 can rotate about the first axis 236, which is perpendicular to the proximal arm 234_2 (i.e., vertical when the proximal arm 234_2 is horizontal). The second part 239 is rotatably coupled to the first part 238 via a second rotary joint (not visible) such that the second part 239 can rotate about the second axis 237, which is perpendicular to the first axis 236 and parallel to the longitudinal dimension 297 of the rail 221. The first part 238 is coupled to the first carriage 226 (or the first carriage 226 is part of the first part 238). As described above in relation to the coupling portions 135_2 of FIGS. 4A-4D, the multiple rotary joints of the coupling portion 235_2 allow the proximal arm 234_2 to be inclined or declined relative to the horizontal plane, which can increase a range of motion of the more distal portions of the manipulator 240.

In addition, each second carriage 227 couples the rail assembly 220 to the platform assembly 210 (e.g., to the middle section 203_3) such that the rail 221 can translate relative to the platform assembly 210 and the support column 202 along the longitudinal dimension 297 of the rail 221. In the embodiment of FIGS. 5-7D, because the rail assembly 220 is coupled to the platform middle section 203_3, the longitudinal dimension 297 of the rail 221 is parallel to the longitudinal dimension 298 of the platform assembly 210 regardless of how the platform assembly 210 is moved or oriented relative to the support column 202.

The one or more second carriages 227 couple the rail 221 to the table assembly 201, specifically to the middle section 203_3 in the embodiment illustrated in FIGS. 3-5D, such that the rail 221 can translate relative to the platform assembly 210 and support column 202 in a direction of the longitudinal dimension 297 of the rail 221. This translation between the rail 221 and the platform assembly 210 and/or support column 202 is provided at least in part by the rail 221 translating relative to the second carriages 227. Because the second carriages 227 are attached to the platform assembly 110, the relative translation between the second carriages 227 and the rail 221 causes translation of the rail 221 relative to the platform assembly 210 (i.e., in a reference frame fixed to the platform assembly 210 or to the ground, the second carriages 227 are seen as stationary and the rail 221 is seen as translating).

FIGS. 7A-7D show the platform assembly 210 and the rail assembly 220 in various configurations illustrative of a range of motion provided by the rail assembly 220 and various ranges of motion of the platform assembly 210 in some embodiments. In FIG. 7A, the second carriage 227 is positioned at a middle portion of the rail 221, such that the rail 221 is situated near the middle sections 203_2 and 203_3 of the platform assembly 210, with some portion of its length extending near section 203_4. In FIG. 7A, the platform assembly 210 is in a neutral position with the manipulators 240 in a deployed state.

In FIG. 7B, the second carriage 227 (not visible in FIG. 7B) is positioned at a head end portion of the rail 221, such that the rail 221 has translated to the right from FIG. 7A and is situated near the foot end portion of the platform 210 (under the middle section 203_3 and the foot section 203_4). FIG. 7B also illustrates optional declined and inclined configurations the middle section 203_2 can be placed in in this positioning of the rail 221 (the declined configuration is illustrated in dashed lines). The positioning of the rail assembly 220 in the configuration of FIG. 7B allows the first end section 203_1 to be declined or inclined (not illustrated) relative to the middle section 203_2, and/or allows the middle section 203_2 to be declined or inclined relative to the middle section 203_3 as illustrated in FIG. 7B, without interference (collision) with the rail assembly 220. In addition, with the rail 221 in the illustrated configuration, additional space is opened up under the first end section 203_1 and/or intermediate section 203_2, which can allow other equipment, such as a C-arm, x-ray equipment, etc., to be positioned near and/or under those sections 203. Moreover, in this configuration of the rail assembly 220, the first carriages 226 can both be positioned near an end of the platform 210 (e.g., a foot end of the platform 210 in FIG. 7B). With the first carriage 226 positioned near an end of the platform 210, the proximal arms 234 can be moved in the nested configuration. As the proximal arms 234 are rotated into the nested configuration, the more distal portions of the manipulators 240 (e.g., intermediate arm 242, distal arm 243, and instrument holder 241) can be swung around a foot end of the platform assembly 210 (i.e., beyond the foot section 203_4) such that the manipulators 240 are positioned along the laterally extending side 209a of the platform assembly 210 instead of along the longitudinally extending side 209b, as shown in FIGS. 7B, 9, and 10. This allows the manipulators 240 to be positioned out of the way of operations that need space along the longitudinally extending sides 209b of the platform assembly 210, such as an operation of transferring a patient from a gurney to the platform assembly 210. For example, FIG. 9 illustrates a state in which a gurney 270 is positioned adjacent a longitudinally extending side 209b of the platform assembly 210. As shown in FIG. 9, because the manipulators 240 are moved to positions along the laterally extending side 209a in the nested configuration, the gurney 270 is able to come right up to the platform assembly 210 flush with a longitudinally extending side 209b thereof without interference from the manipulator 240. Moreover, this positioning of the manipulators 240 can allow for open space along the opposite longitudinally extending side 209b, as well as space around the head and/or foot of the table assembly 201, which can allow personnel to be positioned around the platform 210 to aid in transfers the patient from gurney 270 to platform 210 without concern of collision with the manipulators 240.

In FIG. 7C, the second carriage 227 is positioned at a right end portion of the rail 221, such that the rail 221 is near a left end portion of the platform assembly 210. This configuration of the rail assembly 220 can allow the second end section 203_4 to be declined relative to the middle section 203_3 without collision with the rail assembly 220. FIG. 7C also illustrates the second end section 203_4 in declined and neutral configurations.

In FIG. 7D, inclined (shown in dashed) and declined (shown in solid) configurations of the platform assembly 210 as a whole are illustrated. These configurations can be achieved by rotating the middle section 203_3 relative to the support column about an axis parallel to the lateral dimension 299. As shown in FIG. 7D, the rail assembly 220 and the manipulators 240 move along with the platform assembly 210 in these embodiments because the rail assembly 220 is attached to the platform assembly 210. In other embodiments (not illustrated), the rail assembly 220 can be attached to the support column 202.

Thus, the rail assembly 220 provides a wide range of motion for the manipulators 240, which can allow the manipulators 240 to be positioned nearly anywhere along the longitudinal dimension 298 of the platform assembly 210, including beyond a head or foot end (i.e., beyond laterally extending sides) of the platform assembly so as to clear the entire longitudinal dimension 298 of the platform assembly 210. Moreover, not only does the rail assembly 220 provide a wide range of motion for the manipulators 240, the rail assembly 220 does so while also minimizing interference between the rail assembly 220 and the platform assembly 210, personnel, and other equipment. In particular, the rail assembly 220 is movable between different positions based on the configuration of the platform assembly 210 and/or based on the needs of a particular operation such that the rail 221 is positioned out of the way of the portions of the platform assembly 210, personnel, or other equipment as needed.

The staggered vertical heights of the proximal arms 234 of the manipulators 240, which allows the proximal arms 234 to be vertically nested in a deployed state, can also provide additional benefits. For example, the staggered vertical heights of the proximal arms 234 facilitates an easier and/or more compact stowing of the manipulators 240. For example, as shown in FIG. 5, staggering of the heights of the proximal arms 234 allows them to be oriented at outward angles 180 degrees relative to the rail 221 and vertically overlapping while in the stowed state. This prevents the manipulators 240 from protruding laterally beyond a longitudinal side of the platform 210 when stowed, thus allowing for easier storage and/or movement of the overall table assembly 201 when the manipulators 240 are stowed.

Turning now to FIGS. 8A and 8B, an embodiment of a table-mounted manipulator system 300 (“system 300”) is described below. The system 300 can be used as the system 100, and some components of the system 300 can be used as components of the system 100 described above. Thus, the descriptions of the components of the system 100 above are applicable to the related components of the system 300, and duplicative descriptions of these components are omitted below. The related components of the systems 100 and 300 are given reference numbers having the same right-most two digits—for example, 110 and 310. Although the system 300 is one embodiment of the system 100, the system 100 is not limited to the system 300.

As shown in FIGS. 8A and 8B, the system 300 includes a table assembly 301, a rail assembly 320 coupled to the table assembly, and multiple manipulators 340 coupled to the rail assembly 320. The table assembly 301 includes a platform assembly 310 (also “platform 310”) configured to support the patient or inanimate workpiece, a support column 302 coupled to and supporting the platform assembly 310, and a base (not illustrated) coupled to the support column 302. The platform assembly 310, rail assembly 320, support column 302, and base can be similar to the corresponding components described above in relation to FIGS. 1A-2, and thus duplicative description is omitted. The system 300 also can include a control system (not illustrated), a user input and feedback system (not illustrated), and/or an auxiliary system (not illustrated) similar to those described above in relation to the system 100. In some embodiments, the system 300 is configured as a computer-assisted, teleoperable medical system. In other embodiments, the system 300 is configured as a teleoperable system for use in non-medical contexts.

Each manipulator 340 is configured to carry one more instruments (not illustrated), which can be removably or permanently mounted thereon. In FIGS. 8A and 8B, only a proximal portion of the manipulators 340 is illustrated. A proximal end portion of the manipulators 340 includes a proximal arm 334, which is coupled to the rail 321 of the rail assembly 320 via a coupling portion 335. The coupling portion 335 can be part of the proximal arm 334, or can be a separate part coupled thereto. The coupling portion 335 can include one or more joints (not illustrated) to enable the proximal arm 334 to move relative to the rail 321, including at least a first rotary joint configured to allow for rotation of the proximal arm 334 around an axis 336, which is oriented vertically as illustrated in FIGS. 8A and 8B (i.e., perpendicular to a horizontal plane defined by the lateral and longitudinal dimensions 396 and 397 of the rail 321; in some embodiments, the lateral and longitudinal dimensions 396 and 397 of the rail 321 are parallel to the lateral and longitudinal dimensions 398 and 399 of the platform assembly 310). The coupling portions 335 are movably coupled to the rail 321 (e.g., via first carriages, not illustrated) to allow translation thereof along a longitudinal dimension 397 of the rail 321. The proximal arms 334 are configured to extend horizontally (parallel to a horizontal plane defined by lateral and longitudinal dimensions 396 and 397 of the rail 321). The coupling portions 335 include rotary joints configured to provide for rotation of the proximal arms 334 relative to the rail 321 about axes 336,

FIG. 8A illustrates the system 300 in a state in which the manipulators 340 are deployed and positioned along a longitudinal side 309b of the platform assembly 310. In FIG. 8B, the coupling portions 335 of the manipulators 340 have been moved to be near an end portion of the platform assembly 310 (e.g., a foot end) and the proximal arms 334 thereof have been rotated about respective axes 336 to bring the proximal arm 334 into a nested configuration. Specifically, the proximal arm 334_1 is rotated to an outward angle φ_1 that is equal to or greater than 180 degrees and the proximal arm 334_2 is rotated to an outward angle φ_2 that is equal to 180 degrees. However, unlike in the embodiments illustrated in FIGS. 3A-7D in which the nested configuration included a vertically nested configuration, in the system 300 the nested configuration includes a horizontally nested configuration in which the proximal arms 334 of two adjacent manipulators 340 are arranged side-by-side and overlap one another in a horizontal direction, specifically in a direction aligned with the lateral dimension 396 of the rail 321 (a y-axis direction in FIG. 8B), which in some embodiments is also parallel to the lateral dimension 398 of the platform assembly 310)

To enable the horizontal nesting of the proximal arms 334 as described above, one of the coupling portions 335 can be configured to offset the proximal arm 334 coupled thereto away from the axis of rotation 336. For example, as shown in FIGS. 8A and 8B, the coupling portion 335_2 includes an L-shaped bend 249, which causes the proximal arm 334_2 to be offset from the axis 336. In other words, the coupling portion 335_2 extends from the axis 336 a certain distance in a direction that is different from (e.g., perpendicular to) a direction of extent of the proximal arm 334_2, and then turns (at the bend 249) to begin extending in the direction of the proximal arm 334_2. Because the proximal arm 334_2 is offset from the axis 336, when the proximal arm 334_2 is rotated so as to be parallel to the longitudinal dimension 397 of the rail 327, then in this state the proximal arm 334_2 is offset laterally relative to the rail 221 instead of being aligned with the rail 221. This allows the proximal arm 334_2 to be positioned adjacent to and horizontally overlapping with the proximal arm 334_1, as shown in FIG. 8B. This positioning allows the distal portions of the manipulators 340 to be moved near to the laterally extending side 309a of the platform assembly 310, thus freeing up some space along the longitudinally extending side 309b of the platform assembly 310. However, because the proximal arm portion 334_2 is offset horizontally from the rail 221 in this state, the proximal arm portion 334_2 does protrude somewhat laterally beyond (outwardly of) the rail 221. Thus, while the horizontal nesting can beneficially allow for more space along the longitudinally extending side 309b, in the embodiments of FIGS. 8A and 8B the longitudinally extending side 309b of the platform assembly 310 may not be completely clear of all obstructions in the nested configuration, unlike some embodiments described above which provide for vertical nesting and allow the longitudinally extending side of the platform assembly to be fully cleared. Nevertheless, the partial clearance of the longitudinally extending side 309b of the platform assembly 310 provided by the horizontal nesting can be sufficient for certain tasks. For example, some gurneys can have relatively open regions disposed at certain portions thereof (e.g., under a foot end thereof), and these open regions can align with the partially protruding proximal arm portion 334_2 when the gurney is placed adjacent the platform assembly 310 such that the gurney can be positioned flush or near-flush to the platform assembly 310 notwithstanding the partial protrusion of the proximal arm portion 334_2.

The embodiments described herein may be well suited for use in any of a variety of medical procedures, as described above. Such procedures could be performed, for example, on human patients, animal patients, human cadavers, animal cadavers, and portions or human or animal anatomy. Medical procedures as contemplated herein include any of those described herein and include, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments can also be used for benchtop procedures on non-living material and forms that are not part of a human or animal anatomy. Moreover, some embodiments are also suitable for use in non-medical applications, such as industrial robotic uses, and sensing, inspecting, and/or manipulating non-tissue work pieces. In non-limiting embodiments, the techniques, methods, and devices described herein can be used in, or can be part of, a computer-assisted surgical system employing robotic technology such as the da Vinci® Surgical Systems commercialized by Intuitive Surgical, Inc., of Sunnyvale, California. Those skilled in the art will understand, however, that aspects disclosed herein can be embodied and implemented in various ways and systems, including manually operated instruments and computer-assisted, teleoperated systems, in both medical and non-medical applications. Reference to the da Vinci® Surgical Systems are illustrative and not to be considered as limiting the scope of the disclosure herein.

As used herein and in the claims, terms such as computer-assisted manipulator system, teleoperable manipulator system, or the like should be understood to refer broadly to any system including one or more controllable kinematic structures (“manipulators”) that are movable and controllable at least in part through the aid of an electronic controller (with or without human inputs). Such systems can occasionally be referred to in the art and in common usage as robotically assisted systems or robotic systems. Such systems include systems that are controlled by a user (for example through teleoperation), by a computer automatically (so-called autonomous control), or by some combination of these. In examples in which a user controls at least some of the operations of the manipulator, an electronic controller (e.g., a computer) can facilitate or assist in the operation. The term “computer” as used in “computer-assisted manipulator systems” refers broadly to any electronic control device for controlling, or assisting a user in controlling, operations of the manipulator, and is not intended to be limited to things formally defined as or colloquially referred to as “computers.” For example, the electronic control device in a computer-assisted manipulator system could range from a traditional “computer” (e.g., a general-purpose processor plus memory storing instructions for the processor to execute) to a low-level dedicated hardware device (analog or digital) such as a discrete logic circuit or application specific integrated circuit (ASIC), or anything in between. Further, manipulator systems can be implemented in a variety of contexts to perform a variety of procedures, both medical and non-medical. Thus, although some examples described in greater detail herein can be focused on a medical context, the devices and principles described herein are also applicable to other contexts, such as industrial manipulator systems.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes can be made without departing from the spirit and scope of this description and the claims. In some instances, well-known circuits, structures, and techniques have not been shown or described in detail in order not to obscure the embodiments. Like numbers in two or more figures represent the same or similar elements.

Further, the terminology used herein to describe aspects of the invention, such as spatial and relational terms, is chosen to aid the reader in understanding example embodiments of the invention but is not intended to limit the invention. For example, spatial terms-such as “beneath”, “below”, “lower”, “above”, “upper”, “proximal”, “distal”, “up”, “down”, and the like—may be used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein may need to be interpreted differently in that different reference frame. For example, the direction referred to as “up” in relation to one of the figures may correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items may be posed differently.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled can be electrically or mechanically directly coupled, or they can be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

As used herein, “proximal” and “distal” are spatial/directional terms that describe locations or directions based on their relationship to the two ends of a kinematic chain. “Proximal” is associated with the end of the kinematic chain that is closer to the base or support of the chain, while “distal” is associated with the opposite end of the kinematic chain, which often includes an end effector of an instrument. When used in to refer to locations or to portions of a component, proximal and distal indicate the relative positions of the locations or portions relative to the base of the chain, with the proximal location or portion being closer to the base (closer here referring to proximity along the kinematic chain, rather than absolute distance). When used to refer to a direction, “proximal” refers to directions that point generally from a given location along a kinematic chain towards a more proximal location along the kinematic chain, and “distal” refers to directions that point from the given location towards a more distal location along the kinematic chain.

Further modifications and alternative embodiments will be apparent to those of ordinary skill in the art in view of the disclosure herein. For example, the devices and methods can include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, can be substituted for those illustrated and described herein, parts and processes can be reversed, and certain features of the present teachings can be utilized independently, all as would be apparent to one skilled in the art after having the benefit of the description herein. Changes can be made in the elements described herein without departing from the spirit and scope of the present teachings and following claims.

It is to be understood that the particular examples and embodiments set forth herein are non-limiting, and modifications to structure, dimensions, materials, and methodologies can be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. A teleoperable manipulator system, comprising:
a table assembly comprising a platform configured to support a body;
a rail coupled to the table assembly, wherein the rail comprises lateral and longitudinal dimensions; and
first and second manipulators comprising respective proximal arms coupled to the rail and respective distal portions coupled to the proximal arms and configured to support an instrument mounted thereon,
wherein the proximal arms of the first and second manipulators are translatable relative to the rail along the longitudinal dimension of the rail and are rotatable relative to the rail about one or more axes, and
wherein the proximal arms of the first and second manipulators are positionable in a nested configuration relative to one another in which the proximal arms of the first and second manipulators:
are oriented parallel to the longitudinal dimension of the rail or angled laterally inwardly from the rail, and
at least partially overlap one another in a vertical direction, the vertical direction being perpendicular to a first plane containing the lateral and the longitudinal dimensions of the rail.

2. The system of claim 1,
wherein the proximal arms of the first and second manipulators are positionable in the nested configuration relative to one another while in a deployed state.

3. (canceled)

4. The system of claim 1,
wherein in the nested configuration the proximal arms of the first and second manipulators are parallel to the first plane containing the given direction is perpendicular to the lateral and longitudinal dimensions of the rail.

5. The system of claim 4,
wherein the platform comprising a pair of opposite longitudinally extending sides and a pair of laterally extending sides connecting the longitudinally extending sides at opposite end portions of the platform, and
wherein in the nested configuration and in a deployed state of the first and second manipulators, the respective distal portions of the first and second manipulators are positioned beyond one of the end portions of the platform and adjacent to a laterally extending side of the platform.

6. The system of claim 5,
wherein in the nested configuration no portion of the first or second manipulators protrudes laterally outwardly beyond a longitudinally extending side of the platform.

7. The system of claim 6,
wherein the respective proximal arms of the first and second manipulators, when both are oriented parallel to the first plane, are positioned at different heights relative to the rail.

8. The system of claim 1,
wherein the first and second manipulators further comprise respective coupling portions that rotatably couple the proximal arms to the rail, and
wherein the coupling portion of the first manipulator has a longer height dimension than the coupling portion of the second manipulator.

9. The system of claim 8,
wherein the coupling portion of the first manipulator comprises a first part coupled to the rail and a second part coupled to the first part and to the proximal arm of the first manipulator,
wherein the second part is rotatable relative to the first part about a second axis parallel to the first plane, and
wherein the proximal arm of the first manipulator is rotatable relative to the second part about a first axis, the first axis perpendicular to the first plane.

10. The system of claim 1,
wherein the first and second manipulators further comprise respective coupling portions that rotatably couple the proximal arms to the rail, and
wherein the coupling portion of the first manipulator allows the proximal arm of the first manipulator to rotate around a first axis and around a second axis, wherein, in a state of the proximal arm parallel to the first plane, the first axis is perpendicular to the first plane and the second axis is parallel to the first plane.

11-13. (canceled)

14. The system of claim 1,
wherein in the nested configuration and in a deployed state, the distal portions of the first and second manipulators are positioned higher than the respective proximal arms of the first and second manipulators.

15. The system of claim 1,
wherein the distal portions of the first and second manipulators comprise instrument holders, and
in the nested configuration the instrument holders of the first and second manipulators are positioned higher than the platform.

16. The system of claim 1,
wherein in the nested configuration the proximal arms are positioned adjacent one another at one of the end portions of the platform.

17. The system of claim 1,
wherein the platform comprises an assembly of a plurality of sections movable relative to one another to change a configuration of the platform between a plurality of configurations.

18. The system of claim 17,
wherein the plurality of sections comprise a first end section, one or more middle sections, and a second end section consecutively positioned along a longitudinal dimension of the platform, and
wherein each of the first and second end sections are independently pivotable relative to the one or more middle sections.

19. The system of claim 1, further comprising:
first carriages movably coupling the proximal arms of the first and second manipulators, respectively, to the rail such that the proximal arms are translatable relative to the rail along the longitudinal dimension of the rail.

20. The system of claim 1,
a control system operably coupled to drive the first and second manipulators to position the proximal arms in the nested configuration.

21. The system of claim 1,
wherein table assembly comprises a support column supporting the platform assembly,
wherein the platform is tiltable relative to the support column; and
wherein the rail is configured to tilt along with the platform relative to the support column.

22.-26. (canceled)

27. The system of claim 19,
wherein the rail comprises a first end, a second end, and a straight portion extending linearly between the first end and the second end; and
wherein in the nested configuration the first carriages are positioned on the straight portion of the rail and the proximal arms are oriented parallel to the first plane.

28. The system of claim 27,
wherein the first and second manipulators are positionable between a stowed position and a plurality of deployed positions,
wherein in the stowed position, the first carriages are positioned on the straight portion of the rail, and the first and second manipulators are in the nested configuration and both are positioned under one of the end portions of the platform and do not protrude beyond the laterally and longitudinally extending sides of the platform.

29. A method of operating a teleoperable manipulator system comprising a table assembly having a platform configured to support a body, a rail coupled to the table assembly, and first and second manipulators movably coupled to the rail, the method comprising:
moving respective proximal arms of the first and second manipulators into a nested configuration relative to one another in which the proximal arms of the first and second manipulators:
are oriented parallel to the longitudinal dimension of the rail or angled laterally inwardly from the rail, and
at least partially overlap one another in a vertical direction, the vertical direction being perpendicular to a first plane containing the lateral and the longitudinal dimensions of the rail.
